(19) United States
(12) Patent Application Publication
(10) Pub. No.: US 2016/0346291 A1
(43) Pub. Date: Dec. 1, 2016

(54) COMPOSITIONS AND THERAPEUTIC METHODS FOR ACCELERATED PLAQUE REGRESSION

(71) Applicant: Kenneth Eugene LEBIODA, (US)

(72) Inventor: Kenneth Eugene LEBIODA, (US)

(73) Assignee: Resverlogix Corp, Calgary (CA)

(21) Appl. No.: 14/912,517

(22) PCT Filed: Aug. 21, 2014
(86) PCT No.: PCT/IB2014/002560

§ 371 Date: Feb. 17, 2016

Related U.S. Application Data
(60) Provisional application No. 61/868,382, filed on Aug. 21, 2013.

(51) Int. Cl. A61K 31/5377; A61K 31/4375; A61K 31/517; A61K 31/519; A61K 31/505; A61K 31/47

(52) U.S. Cl. CPC A61K 31/5377; A61K 31/505; A61K 31/47; A61K 31/517; A61K 31/519; A61K 31/4375

(57) ABSTRACT

The invention comprises methods for treating and/or preventing cardiovascular, cholesterol, and lipid related disorders, including atherosclerosis, through-co-administration of therapeutically effective amounts of a compound of Formula I or a pharmaceutically acceptable salt thereof and rosuvastatin or pravastatin or a pharmaceutically acceptable salt of rosuvastatin or pitavastatin. The invention further provides compositions comprising a therapeutically effective amount of a compound of Formula I or a pharmaceutically acceptable salt thereof and a therapeutically effective amount of or pitavastatin or a pharmaceutically acceptable salt of rosuvastatin or pitavastatin.

This application claims priority from U.S. Provisional Patent Application No. 61/868,382, filed Aug. 21, 2013, which is hereby incorporated by reference in its entirety.

The present disclosure relates to methods of treating and/or preventing atherosclerosis and related disorders through combination therapy with rosuvastatin [(3R,5S,6E)-7-[4-(4-fluorophenyl)-2-(N-methylmethanesulfonamido)-6-(propan-2-yl)pyrimidin-5-yl]-3,5-dihydroxyhept-6-enoic acid] or a pharmaceutically acceptable salt thereof, or pitavastatin [(3R,5S,6E)-7-[2-cyclopropyl-4-(4-fluorophenyl)quinolin-3-yl]-3, 5-dihydroxhept-6-enoic acid] or a pharmaceutically acceptable salt thereof and a compound of Formula I or a pharmaceutically acceptable salt thereof that upregulates expression of apolipoprotein A-I.

Cardiovascular disease (CVD) is the leading cause of morbidity and mortality in the Western world. An underlying cause of CVD is hardening and narrowing of the arteries due to atherosclerosis—the build-up of cholesterol in the arteries that forms an atherosclerotic plaque. It is present in all vascular beds of the body including, but not limited to, coronary, brain and peripheral (legs and arms). Atherosclerosis is a leading driver of diseases such as coronary heart disease, stroke, dementia, cognitive impairment, kidney disease, and peripheral artery disease.

It is well accepted that high serum levels of low-density lipoproteins (LDL) are responsible for the initiation and progression of atherosclerosis, while high-serum levels of high-density lipoproteins (HDL) are responsible for preventing or even regressing atherosclerosis. T. Gordon et al., “High Density Lipoprotein as a Protective Factor Against Coronary Heart Disease: The Framingham Study” Am. J. Med. 62:707-714 (1977), G. Assmann et al., “The Münster Heart Study (PROCAM). Results of Follow-up at 8 Years” Eur. Heart J. 19(A):A2-A11 (1998).

A variety of therapeutic options are currently employed in the treatment of CVD and conditions associated with CVD and aberrant cholesterol levels (i.e., cholesterol- and lipid-related disorders). Many of these therapeutic options function by lowering cholesterol levels, particularly LDL levels. Among the most popular and effective of these therapeutic options are statins, a class of compounds that inhibit cholesterol biosynthesis and prevent the build-up of arterial plaque. Statin administration has been shown to lower LDL levels and to substantially reduce coronary events and death. T. R. Pedersen et al., “Randomised Trial of Cholesterol Lowering in 4444 Patients with Coronary Heart Disease: The Scandinavian Simvastatin Survival Study (4S)” Lancet 344:1383-1389 (1994). However, statin therapy alone is insufficient to completely treat CVD and substantial residual risk remains. P. Libby, “The Forgotten Majority: Unfinished Business in Cardiovascular Risk Reduction” J. Am. Coll. Cardiol. 46(7):1225-1228 (2005). As a result, there is an important need to develop new therapeutic strategies that complement current therapies to achieve more effective reduction in cardiovascular events.

One recent therapeutic approach has been to elevate the levels of HDL or ApolipoproteinA-I (ApoA-I)—the major protein component of HDL—to promote reverse cholesterol transport (RCT). The protective effect of HDL in atherosclerosis, either by RCT, suppressing vascular-LDL accumulation, inflammation, oxidation, endothelial damage, and thrombosis [E. A. Fisher et al., “High-Density Lipoprotein Function, Dysfunction, and Reverse Cholesterol Transport” Arterioscler. Thromb. Vasc. Biol. 32:2813-2820 (2012)], has supported the need for HDL-raising therapies to address this residual risk. Therapeutic options to increase HDL include niacin, fibrates, ApoA-I mimetics, CETP inhibitors, etc. Furthermore, it is becoming increasingly evident that HDL particle functionality is as important as HDL levels. A. V. Khera et al., “Cholesterol Efflux Capacity, High-Density Lipoprotein Function, and Atherosclerosis” N. Engl. J. Med. 364:127-35 (2011). As a result, there is an important need to develop new therapeutic strategies that increase HDL, but in particular, HDL functionality to achieve more effective reduction of cardiovascular events.

Another strategy to elevate levels of HDL and reduce the risk of atherosclerosis or other cholesterol- or lipid-related disorders, such as e.g., cardiovascular disease, is to increase expression of ATP-binding cassette transporter A1 (ABCA1). ABCA1 expression in the liver was shown to be necessary for HDL formation. Inactivation of the ABCA1 gene in mice leads to a severe HDL deficiency (Aiello, R. J., et al. Arterioscler. Thromb. Vasc. Biol. (2003) 23, 972-980), and targeted disruption of the ABCA1 gene in mouse hepatocytes reduces plasma HDL level by 80% (Lee, J. Y., Parks, J. S., Cur. Opin. Lipidol. (2005) 16, 19-25). Conversely, overexpression of ABCA1 in mouse liver markedly increases plasma HDL level (Wellington, C. L., et al. J. Lipid Res. (2003) 44, 1470-1480). Both rosuvastatin (Shimizu. T et al. Circulation (2011) 124, A11181) and pitavastatin (Kobayashi, M. et al. Eur. J. Pharmacol. (2011) 662, 9-14) have been shown to increase expression of ABCA1. In contrast, for example, atorvastatin appears to inhibit ABCA1 expression (Qiu, G., Hill, J. S. J. Cardiovasc. Pharmacol (2008) 51, 388-395).

Arterial wall imaging has been increasingly incorporated into clinical development programs for therapies that treat vascular diseases. The development of intravascular ultrasound (IVUS) permits imaging within the coronary and other arteries with high-frequency ultrasound transducers. This generates high-resolution images of the entire thickness of the artery wall, allowing visualization of the full extent of atherosclerosis. As a result, IVUS permits precise quantitation of the burden of atherosclerotic plaque and therefore provides an opportunity to evaluate the impact of medical therapies on disease progression by measuring percent atheroma volume (PAV) or total atheroma volume (TAV) before and after treatment. S. Brugaletta et al., “NIRS and IVUS for Characterization of Atherosclerosis in Patients Undergoing Coronary Angiography” JACC: Cardiovasc Imaging 4(6):647-655 (2011). Clinical studies that employ IVUS are able to elucidate the beneficial impact (prevention or regression) of therapies on the burden of coronary atherosclerosis. S. J. Nicholls et al., “Relationship Between Cardiovascular Risk Factors and Atherosclerotic Disease Burden Measured by Intravascular Ultrasound” J. Am. Coll. Cardiol. 47(10):1967-1975 (2006). More recently, findings from clinical trials that employed serial IVUS imaging have provided important insights into the benefits of stalin therapies and HDL therapies.

A post-hoc analysis of four IVUS clinical studies of 1455 CVD patients treated with either pravastatin 40 mg, atorvastatin 80 mg, enalapril 20 mg, amlopipine 10 mg, pactimibe 100 mg or rosuvastatin 40 mg illustrated the beneficial impact of statins on plaque progression. This study showed that the natural annual progression of PAV is, on average, about 0.6%. S. J. Nicholls et al., “Statins, High-Density Lipoprotein Cholesterol and Regression of Coronary Atherosclerosis” JAMA 297(5):499-508 (2007). In one trial (REVERSAL). 2 years of treatment with the highest dose of pravastatin (40 mg daily) was compared with intensive dose atorvastatin (80 mg daily). The results showed that both pravastatin (median change in PAV=+1.6%) and atorvastatin did not regress atherosclerosis (median change in PAV=+0.2%). S. E. Nissen et al., “Effect of Intensive Compared with Moderate Lipid-Lowering Therapy on Progression of Coronary Atherosclerosis: A Randomized Controlled Trial” JAMA 291(9): 1071-1080 (2004).

In another trial (SATURN), 2 years of treatment with the highest dose of rosuvastatin (40 mg daily) was compared to the highest dose of atorvastatin (80 mg daily). Both treatment regimens showed regression of atherosclerosis (median change in PAV of −1.22% and −0.99%, respectively). S. J. Nicholls et al., “Effect of Two Intensive Statin Regimens on Progression of Coronary Disease” N. Engl. J. Med. 365:2078-2087 (2011).

In another trial (ASTEROID), 2 years of treatment with maximum dose of rosuvastatin (40 mg daily) showed an overall regression of atherosclerosis (median change PAV=−0.79%). In a subgroup of patients with low HDL, patients most at risk of a cardiovascular event, and defined as patients with an average HDL-C during treatment with less than 40 mg/dL, treatment with rosuvastatin showed a median decrease in PAV from baseline of −1.3%. S. E. Nissen et al., “Effect of Very High-intensity Statin Therapy on Regression of Coronary Atherosclerosis. The ASTEROID Trial” JAMA 295(13):1556-1563 (2006). Maximum doses of statins have been shown to prevent and regress atherosclerosis over a long (2 years+) period of time. However, these doses are often not tolerated by patients and cause several side effects including myopathy and renal events. V. M. Alla et al., “A Reappraisal of the Risks and Benefits of Treating to Target with Cholesterol Lowering Drugs” Drugs 73(10):1025-1054 (2013). Thus, there is a need to treat patients with lower and better tolerated doses of statins while regressing atherosclerosis over a shorter period of time.

In the MILANO trial, 6 weeks of treatment with ApoA-I Milano formulated with phospholipids and infused once per week at concentrations of 15 and 45 mg/kg body weight showed a combined PAV regression of −1.06% from baseline. S. E. Nissen et al., “Effect of Recombinant ApoA-I Milano on Coronary Atherosclerosis in Patients with Acute Coronary Syndromes: A Randomized Controlled Trial” JAMA 290(17):2292-2300 (2003). In the ERASE trial, wild type ApoA-I/phospholipid particles were infused once weekly for 6 weeks, and similar results were found. J. C. Tardif et al., “Effects of Reconstituted High-Density Lipoprotein Infusions on Coronary Atherosclerosis: A Randomized Controlled Trial” JAMA 297(15):1615-1682 (2007). This illustrated that HDL therapies have a potentially more potent effect in reducing atherosclerosis and a quicker onset of action than statins. This latter aspect is particularly important in secondary prevention, because a second cardiovascular event is known to occur shortly after a first cardiovascular event in patients with CVD.

Given this background the link between atherosclerosis and vascular, particularly cardiovascular, events is beyond doubt. As a consequence, treatments that regress atherosclerosis are of great interest in terms of preventing CVD and other vascular events associated with cholesterol- and/or lipid-related disorders in high risk patients. Thus, there is a continuing need for treatments that regress atherosclerosis because they are crucial to preventing cholesterol- and lipid-related diseases and disorders.

There is also a need for treatment and/or prevention of atherosclerosis and cholesterol- or lipid-related disorders that allows for maximal benefits with reduced dosages of statin drugs. All statins are associated with certain undesirable side effects, including, e.g., muscle aches and/or weakness, muscle damage (rhabdomyoloysis), digestive problems (such as nausea, gas, diarrhea, constipation, abdominal pain), headaches, memory loss or confusion, increased risk of developing type 2 diabetes, liver damage, and in some cases, kidney failure. The risk of incurring any of these side effects increases with an increased dose of the statin.

The present invention satisfies both of these needs by providing compositions comprising rosuvastatin [(3R,5S,6E)-7-[4-(4-fluorophenyl)-2-(N-methylmethanesulfonamido)-6-(propan-2-yl)pyrimidin-5-yl]-3,5-dihydroxyhept-6-enoic acid] or a pharmaceutically acceptable salt thereof, or pitavastatin [(3R,5S,6E)-7-[2-cyclopropyl-4-(4-fluorophenyl)quinolin-3-yl]-3,5-dihydroxhept-6-enoic acid] or a pharmaceutically acceptable salt thereof, and a compound of Formula I or a pharmaceutically acceptable salt thereof, as well as methods of treatment with those compositions and/or combinations of rosuvastatin (or a pharmaceutically acceptable salt thereof) and a compound of Formula I (or a pharmaceutically acceptable salt thereof) that promote regression of atherosclerosis and/or maximize the benefits of statins, allowing lower doses to be administered and consequently minimizing unwanted side effects of statin therapy.

### Compounds of Formula I

Therapeutic strategies that increase endogenous synthesis of ApoA-I remain of great interest in the development of new therapies to promote HDL function and RCT. The compounds of Formula I are members of a novel class of small molecules that increase ApoA-I levels by transcriptional upregulation. By increasing ApoA-I, treatment with a compound of Formula I or a pharmaceutically acceptable salt thereof may enable the removal of atherosclerotic plaque via increasing HDL and RCT, the natural process through which atherosclerotic plaque is transported out of the arteries and removed from the body by the liver.

In vitro and in vivo certain compounds of Formula I have been shown to increase plasma levels of ApoA-I and HDL, and functional particles of HDL including prebeta-HDL and alpha-HDL particles. See, e.g., D. Bailey et al., “RVX-208: a small molecule that increases apolipoprotein A-I and high-density lipoprotein cholesterol in vitro and in vivo” J. Am. Coll. Cardiol. 55(23):2581-2589 (2010). These data demonstrate that compounds of Formula I can induce “functional HDL,” which may have utility in the treatment of cardiovascular disease.

Compounds of Formula I have previously been described in U.S. Pat. No. 8,053,440, incorporated herein by reference. Compounds of Formula I include:

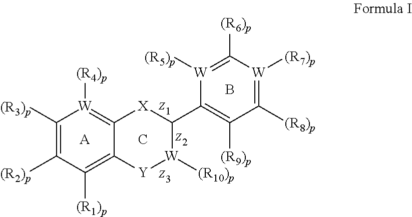

wherein:

X is N;

Y is CO;

R₁ and R₃ are each independently selected from alkoxy, alkyl, amino, halogen, and hydrogen;

R₂ is selected from alkoxy, alkyl, alkenyl, alkynyl, amide, amino, halogen, and hydrogen;

R₆ and R₈ are each independently selected from alkyl, alkoxy, amino, halogen, and hydrogen;

R₅ and R₉ are each hydrogen;

R₇ is selected from amino, amide, alkyl, hydroxyl, and alkoxy;

R₁₀ is hydrogen;

each W is independently selected from C and N, wherein if W is N, then p is 0 or 1, and if W is C, then p is 1;

for W—(R₁₀)ₚ, W is N and p is 1;

for W—(R₇)ₚ, W is C and p is 1;

for W—(R₄)ₚ, W is C, p is 1 and R₄ is H, or W is N and p is 0;

for (R₁)ₚ, p is 1;

for (R₂)ₚ, p is 1;

for (R₃)ₚ, p is 1;

for (R₆)ₚ, p is 1;

for (R₈)ₚ, p is 1;

for (R₉)ₚ, p is 1;

Z₁ is a double bond, and Z₂ and Z₃ are each a single bond;

with the proviso that if R₁ is hydrogen, then R₃ is alkoxy;

with the proviso that if R₃ is hydrogen, then R₁ is selected from amino and alkoxy;

with the proviso that if R₇ is selected from alkyl, hydroxyl, and alkoxy, then at least one of R₆ and R₈ is independently selected from alkyl, alkoxy, amino, and halogen.

In an exemplary embodiment, the compound of Formula I is RVX-208, which has the chemical name [2-(4-(2-hydroxyethoxy)-3,5-dimethylphenyl)-5,7-dimethoxyquinazolin-4(3H)-one] and the structural formula:

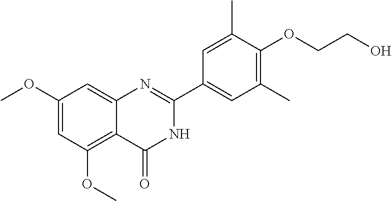 [SMILES: COc1cc(OC)c2c(=O)[nH]c(-c3cc(C)c(OCCO)c(C)c3)nc2c1]

The empirical formula for RVX-208 is C₂₀H₂₂N₂O₅ and the molecular weight is 370.41.

### Rosuvastatin

The calcium salt of rosuvastatin (rosuvastatin calcium, marketed as CRESTOR®) is a synthetic lipid-lowering agent for oral administration. The chemical name for rosuvastatin calcium is bis[(E)-7-[4-(4-fluorophenyl)-6-isopropyl-2-[methyl(methylsulfonyl)amino] pyrimidin-5-yl](3R,5S)-3,5-dihydroxyhept-6-enoic acid] calcium salt. The structure of rosuvastatin calcium is:

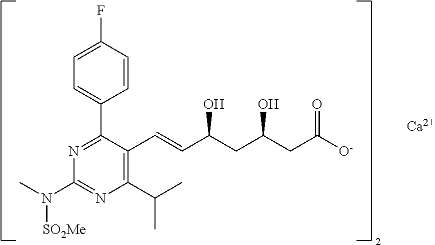

The empirical formula of rosuvastatin calcium is (C₂₂H₂₇FN₃O₆S)₂Ca and the molecular weight is 1001.14.

Rosuvastatin, and it's pharmaceutically acceptable salts, particularly it's calcium salt, are selective inhibitors of HMG-CoA reductase, the rate-limiting enzyme that converts 3-hydroxy-3-methylglutaryl coenzyme A to mevalonate, a precursor of cholesterol. In vivo studies have shown that rosuvastatin, particularly rosuvastatin calcium produces its lipid-modifying effects by increasing the number of hepatic LDL receptors on the cell-surface to enhance uptake and catabolism of LDL.

### Pitavastatin

Pitavastatin (also known as LIVALO®) is another synthetic HMG-CoA reductase inhibitor that, like rosuvastatin, is typically administered as a calcium salt. The chemical name for pitavastatin is (3R,5S,6E)-7-[2-cyclopropyl-4-(4-fluorophenyl)quinolin-3-yl]-3,5-dihydroxyhept-6-enoic acid. Pitavastatin has the following structural formula:

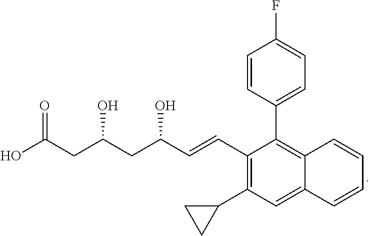 [SMILES: O=C(O)C[C@H](O)C[C@H](O)/C=C/c1c(C2CC2)cc2ccccc2c1-c1ccc(F)cc1]

The empirical formula for pitavastatin is C₃₅H₂₄FNO₄ and the molecular weight is 421.461.

A 6-month intervention study in patients with a history of coronary artery disease treated with RVX-208 and standard-of-care medicines, including one of two statins, rosuvastatin or atorvastatin, demonstrated statistically significant improvement in atherosclerosis in the coronary arteries of study participants as assessed by intravascular ultrasound (IVUS). The study also demonstrated a reduction in the incidence of major adverse vascular (e.g., cardiovascular) events. The effects of treatment with RVX-208 and rosuvastatin were surprisingly and significantly better than the effects of treatment with rosuvastatin or atorvastatin alone, or with RVX-208 and atorvastatin.

### BRIEF DESCRIPTION OF THE FIGURES

FIG. 1 shows the median change in percent atheroma volume (PAV) in patients dosed with RVX-208+atorvastatin who began the study with below median HDL (lane 1) or above median HDL (lane 3); and in patients dosed with RVX-208+rosuvastatin in patients who began the study with below median HDL (lane 2) or above median HDL (lane 4). Median Baseline HDL=39 mg/dL. N refers to number of patients. Rosuvastatin doses=5, 10, or 20 mg. Atorvastatin doses=10, 20, or 40 mg.

FIG. 2 shows the median change in percent atheroma volume (PAV) in patients dosed with RVX-208+any concentration of rosuvastatin and patients dosed with RVX-208+any concentration of atorvastatin (lane 1); in patients dosed with RVX-208+specified concentrations rosuvastatin (regardless of HDL values at initiation of the study) (lanes 2 and 3); and in patients dosed with RVX-208+specified concentrations of rosuvastatin who began the study with below median HDL (lanes 4 and 5) Median Baseline HDL=39 mg/dL. N refers to number of patients.

FIG. 3 shows the median change in total atheroma volume (TAV) in patients dosed with RVX-208+any concentration of rosuvastatin and patients dosed with RVX-208+any concentration of atorvastatin (lane 1), in patients dosed with RVX-208+specified concentrations of rosuvastatin (regardless of HDL values at initiation of the study) (lanes 2 and 3); and in patients dosed with RVX-208+specified concentrations of rosuvastatin who began the study with below median HDL. Median Baseline HDL=39 mg/dL. N refers to number of patients.

FIG. 4 shows the median change in percent atheroma volume (PAV) in patients dosed with RVX-208+any concentration of rosuvastatin and patients dosed with RVX-208+any concentration of atorvastatin (lane 1); in patients dosed with placebo+any concentration of rosuvastatin and patients dosed with placebo+any concentration of atorvastatin (lane 2); and in patients dosed with RVX-208+specified concentrations of rosuvastatin or placebo+rosuvastatin (regardless of initial HDL values) (lanes 3-6); and in patients dosed with RVX-208+specified concentrations of rosuvastatin or placebo+rosuvastatin in patients with below median HDL (lanes 7-12). Median Baseline HDL=39 mg/dL. N refers to number of patients.

FIG. 5 shows the median change in total atheroma volume (TAV) in patients dosed with RVX-208+any concentration of rosuvastatin and patients dosed with RVX-208+any concentration of atorvastatin (lane 1); in patients dosed with placebo+any concentration of rosuvastatin and patients dosed with placebo+any concentration of atorvastatin (lane 2); and in patients dosed with RVX-208+specified concentrations of rosuvastatin or placebo+rosuvastatin (regardless of initial HDL values) (lanes 3-6); and in patients dosed with RVX-208+specified concentrations of rosuvastatin or placebo+rosuvastatin in patients with below median HDL (lanes 7-12). Median Baseline HDL=39 mg/dL. N refers to number of patients.

FIG. 6A shows the percentage of major adverse vascular events (MAVE) in patients receiving RVX-208+rosuvastatin as compared to patients receiving rosuvastatin alone. FIG. 6B shows the percentage of major adverse vascular event (MAVE) in patients receiving RVX-208+atorvastatin as compared to patients receiving atorvastatin alone. Rosuvastatin doses=5, 10, or 20 mg. Atorvastatin doses=10, 20, or 40 mg.

FIG. 7 shows the percentage of major adverse vascular event (MAVE) in patients dosed with rosuvastatin alone and patients dosed with atorvastatin alone (lane 1); in patients dosed with RVX-208+rosuvastatin and patients dosed with RVX-208+atorvastatin (lane 2); in patients receiving placebo+rosuvastatin who began the study with below median HDL (lane 3); and in patients dosed with RVX-208+rosuvastatin who began the study with below median HDL. Median Baseline HDL=39 mg/dL. N refers to number of patients. Rosuvastatin doses=5, 10, or 20 mg. Atorvastatin doses=10, 20, or 40 mg.

FIG. 8 shows the median change in percent atheroma volume (PAV) in patients who began the study with below median HDL dosed with placebo+rosuvastatin (lane 1); or dosed with RVX-208+rosuvastatin at various dosages (lanes 2-4). Median Baseline HDL=39 mg/dL. N refers to number of patients.

### DETAILED DESCRIPTION OF EMBODIMENTS

A 6-month intervention study with a compound of Formula I, RVX-208 and standard-of-care medicines, rosuvastatin or atorvastatin, was conducted. The inclusion criteria for the clinical trial was men with a baseline HDL<40 and women with a baseline HDL<45, both of which are considered low according to clinical guidelines. The study shows that administration of a compound of Formula I. RVX-208 and rosuvastatin results in statistically significant improvements in coronary IVUS atheroma measurements. The study also demonstrates a reduction of the incidence of major adverse vascular events. Surprisingly, it was found that patients treated with the combination of a compound of Formula I, RVX-208 and rosuvastatin showed more pronounced and rapid (6 months) regression of atherosclerosis.

The 6-month effect of RVX-208 and rosuvastatin was quantified using IVUS on median total atheroma volume (TAV) and percent atheroma volume (PAV). The combination therapy not only stopped progression of atherosclerosis but also resulted in significant regression. The highest amount of regression was observed when RVX-208 (200 mg)+rosuvastatin (20 mg) was administered as a combined dose, resulting in TAV of −15.94 mm³ and PAV of −2.04% (n=16).

This synergistic regression effect on IVUS atherosclerosis burden markers—PAV and TAV—in all patients treated with RVX-208 and rosuvastatin, but particularly in patients with a baseline HDL<39 mg/dL—in treatment duration of 6 months was unexpected and surprising. It is even more surprising in view of the fact that a similar effect was not seen with the combination of RVX-208 and atorvastatin. Reduced CVD events in patients with low baseline HDL (men with HDL<40 and women with HDL<45) when treated with both RVX-208 and rosuvastatin were also observed and were consistent with the strong regression of atherosclerosis seen.

Thus, the present invention provides methods of treating and/or preventing atherosclerosis disease and other cholesterol- or lipid-related disorders, by co-administering a therapeutically effective amount of a compound of Formula I or a pharmaceutically acceptable salt thereof in combination with rosuvastatin or pitavastatin or a pharmaceutically acceptable salt of rosuvastatin or pitavastatin. Although bis((E)-7-[4-(4-fluorophenyl)-6-isopropyl-2-[methyl(methylsulfonyl)amino]pyrimidin-5-yl](3R,5S)-3,5-dihydroxyhept-6-enoic acid) and (3R,5S,6E)-7-[2-cyclopropyl-4-(4-fluorophenyl)quinolin-3-yl]-3,5-dihydroxyhept-6-enoic acid are normally formulated as calcium salts, other salt forms are contemplated for use in the compositions and combination therapies of the invention.

References to atherosclerosis and other cholesterol-, or lipid-related disorders are meant to include diseases and disorders that are affected by or associated with aberrant levels of LDL or HDL or result from the build up of plaque in the blood vessels. These diseases and disorders impact the circulatory system, and include cardiovascular diseases, peripheral vascular diseases (or peripheral artery diseases), renal bed vascular diseases, and cerebrovascular diseases. Exemplary diseases and disorders that may be treated with the compositions and combinations of the invention, include, but are not limited to acute coronary syndrome, angina, arteriosclerosis, atherosclerosis, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteirnemia, hypertriglyceridemia, hyperbetalipoproteinernia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, transitory ischemic attacks, and lipoprotein abnormalities associated with Alzheimer's disease, obesity, diabetes mellitus, syndrome X, impotence, multiple sclerosis, Parkinson's diseases and inflammatory diseases.

As used herein, “treatment” or “treating” refers to an amelioration of a disease or disorder, or at least one discernible symptom thereof. In one embodiment. “treatment” or “treating” refers to an amelioration of at least one measurable physical parameter, not necessarily discernible by the subject. In another embodiment, “treatment” or “treating” refers to inhibiting the progression of a disease or disorder, either physically, e.g., stabilization of a discernible symptom, physiologically, e.g., stabilization of a physical parameter, or both. In another embodiment, “treatment” or “treating” refers to delaying the onset of a disease or disorder. For example, treating a cholesterol disorder may comprise decreasing blood cholesterol levels.

As used herein, “prevention” or “preventing” refers to a reduction of the risk of acquiring a given disease or disorder or a symptom of such disease or disorder.

“Patient” refers to an animal, such as a mammal, that has been or will be the object of treatment, observation, or experiment. The methods described herein may be useful for both human therapy and veterinary applications. In one embodiment, the subject is a human.

A “major adverse vascular event” (MAVE) refers to adverse events caused by disease processes generally affecting the cardiovascular, cerebrovascular, renal bed vascular diseases, and/or peripheral vascular systems. These events include, but are not limited to death, myocardial infarction, stroke, revascularization intervention (such as, e.g., implanting a stent), critical limb ischemia, acute coronary syndrome, heart failure, and vascular-related hospitalization.

The term “pharmaceutically acceptable salt” refers to any salt of a compound of Formula I, rosuvastatin, or pitavastatin that retains its biological properties and which is not toxic or otherwise undesirable for pharmaceutical use. Pharmaceutically acceptable salts may be derived from a variety of organic and inorganic counter-ions well known in the art and include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like. Pharmaceutically acceptable salts further include, by way of example only, sodium, potassium, calcium, magnesium, ammonium, tetraalkylammonium and the like, and when the compound contains a basic functionality, salts of non-toxic organic or inorganic acids, such as hydrochloride, hydrobromide, tartrate, mesylate, besylate, acetate, maleate, oxalate and the like.

The term “co-administering” and its cognates, as used herein refers to the administration of a compound of Formula I or a pharmaceutically acceptable salt thereof and rosuvastatin or pitavastatin or a pharmaceutically acceptable salt of rosuvastatin or pitavastatin together as part of a single dosage form (such as a composition comprising both a compound of Formula I (or a pharmaceutically acceptable salt thereof) and rosuvastatin or pitavastatin or a pharmaceutically acceptable salt of rosuvastatin or pitavastatin) or as separate, multiple dosage forms. Alternatively, a compound of Formula I (or a pharmaceutically acceptable salt thereof) may be administered prior to, consecutively with, or following the administration of rosuvastatin or pitavastatin (or a pharmaceutically acceptable salt thereof, such as, e.g., a calcium salt). In such combination therapy treatment, both the compound of Formula I (or a pharmaceutically acceptable salt thereof) and rosuvastatin or pitavastatin (or a pharmaceutically acceptable salt thereof, such as, e.g., a calcium salt) are administered by conventional methods. The co-administration of a compound of Formula I (or a pharmaceutically acceptable salt thereof) and rosuvastatin or pitavastatin (or a pharmaceutically acceptable salt thereof, such as, e.g., a calcium salt) to a patient does not preclude the separate administration of either therapeutic agent, any other therapeutic agent to a patient at another time during a course of treatment.

Thus, one aspect of the invention provides compositions comprising both a compound of Formula I (or a pharmaceutically acceptable salt thereof) and rosuvastatin or pitavastatin (or a pharmaceutically acceptable salt of rosuvastatin or pitavastatin, such as, e.g., a calcium salt) together with one or more pharmaceutically acceptable carriers, diluents, and/or excipients. Another aspect of the invention provides separate dosage forms of a compound of Formula I (or a pharmaceutically acceptable salt thereof) and rosuvastatin or pitavastatin (or a pharmaceutically acceptable salt of rosuvastatin or pitavastatin, such as, e.g., a calcium salt), wherein the compound of Formula I (or a pharmaceutically acceptable salt thereof) and rosuvastatin or pitavastatin (or a pharmaceutically acceptable salt of rosuvastatin or pitavastatin, such as, e.g., a calcium salt) are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously). In another aspect of the invention, a pharmaceutical composition comprising a compound of Formula I (or a pharmaceutically acceptable salt thereof) and one or more pharmaceutically acceptable carriers, diluents, and/or excipients is provided in addition to rosuvastatin or pitavastatin (or a pharmaceutically acceptable salt of rosuvastatin or pitavastatin, such as, e.g., a calcium salt) for use in therapy, in particular for the treatment of atherosclerosis.

### Pharmaceutical Compositions

In certain embodiments, the compound of Formula I (or a pharmaceutically acceptable salt thereof) and rosuvastatin or pitavastatin (or a pharmaceutically acceptable salt of rosuvastatin or pitavastatin, such as, e.g., a calcium salt) are formulated together or separately for oral administration. Formulations suitable for oral administration may be presented in discrete units, such as capsules, cachets, lozenges, tablets, or patches, each containing a predetermined amount of a compound of the present disclosure as powder or granules; as a solution or a suspension in an aqueous or non-aqueous liquid; or as an oil-in-water or water-in-oil emulsion. Such formulations may be prepared by any suitable method of pharmacy which includes the step of bringing into association at least one compound of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution, ointment, or suspension. In general, suitable formulations may be prepared by uniformly and intimately admixing at least one active compound of the present disclosure with a liquid or finely divided solid carrier, or both, and then, if necessary, shaping the product. For example, a tablet may be prepared by compressing or molding a powder or granules of at least one compound of the present disclosure, which may be optionally combined with one or more accessory ingredients. Compressed tablets may be prepared by compressing, in a suitable machine, at least one compound of the present disclosure in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the present disclosure is moistened with an inert liquid diluent.

Formulations suitable for buccal (sub-lingual) administration include lozenges comprising at least one compound of the present disclosure in a flavored base, usually sucrose and acacia or tragacanth, and pastilles comprising the at least one compound in an inert base such as gelatin and glycerin or sucrose and acacia.

The amount of active compound administered may be dependent on the subject being treated, the subject's weight, the manner of administration and the judgment of the prescribing physician. For example, a dosing schedule may involve the daily or twice-daily administration of the encapsulated compound or compounds at a dosage of about 100-300 mg of a compound of formula I (or a pharmaceutically acceptable salt thereof) with 5.0-20 mg of rosuvastatin (or a pharmaceutically acceptable salt thereof, such as, e.g. rosuvastatin calcium) or 1.0-4.0 mg of pitavastatin (or a pharmaceutically acceptable salt thereof, such as, e.g., pitavastatin calcium).

In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Specific embodiments of the invention comprise co-administration of 100-300 mg/day of the compound of Formula I (or a pharmaceutically acceptable salt thereof) and 5-20 mg/day of rosuvastatin (or a pharmaceutically acceptable salt thereof, such as, e.g., a calcium salt) or 0.5-4 mg/day pitavastatin (or a pharmaceutically acceptable salt of pitavastatin, such as, e.g., a calcium salt). These dosages of the compound of Formula I and rosuvastatin or pitavastatin (or a pharmaceutically acceptable salt of rosuvastatin or pitavastatin, such as, e.g., a calcium salt) may be administered once a day or divided for twice a day administration as a single composition. Alternatively, the daily dosages of a compound of Formula I (or a pharmaceutically acceptable salt thereof) and rosuvastatin or pitavastatin (or a pharmaceutically acceptable salt of rosuvastatin or pitavastatin, such as, e.g., a calcium salt) may be administered as separate compositions, once a day or divided for twice a day administration. In such case, the compositions may be administered simultaneously, or sequentially. In some embodiments, the dosage of a compound of Formula I (or pharmaceutically acceptable salt thereof) may be administered twice a day, while the rosuvastatin or pitavastatin (or a pharmaceutically acceptable salt of rosuvastatin or pitavastatin, such as, e.g., a calcium salt) may be administered once a day. In some embodiments, the dosage of the compound of Formula I (or a pharmaceutically acceptable salt thereof) is 100 mg, 150 mg, 200 mg, 250 mg, or 300 mg per day and the dosage of rosuvastatin (or a pharmaceutically acceptable salt thereof, such as, e.g., rosuvastatin calcium) is 5 mg, 7.5 mg, 10 mg, 12.5 mg, 15 mg, or 20 mg per day or the dosage of pitavastatin (or a pharmaceutically acceptable salt thereof, such as pitavastatin calcium) is 0.5 mg, 1.0 mg, 1.5 mg, 2.0 mg, 2.5 mg, 3.0 mg or 4.0 mg per day.

A specific embodiment of the invention provides a pharmaceutical composition comprising 100 mg of a compound of Formula I (or a pharmaceutically acceptable salt thereof) and 5 mg, 10 mg, 15 mg, or 20 mg of rosuvastatin (or a pharmaceutically acceptable salt thereof, such as, e.g., rosuvastatin calcium). In an alternate embodiment, the 100 mg of the compound of Formula I (or a pharmaceutically acceptable salt thereof) and the 5 mg, 10 mg, 15 mg, or 20 mg of rosuvastatin are in separate compositions. In certain embodiments, the pharmaceutical composition comprises 200 mg of a compound of Formula I or a pharmaceutically acceptable salt thereof and 5 mg, 10 mg, 15 mg, or 20 mg of rosuvastatin (or a pharmaceutically acceptable salt thereof, such as, e.g., rosuvastatin calcium). In an alternate embodiment, the 200 mg of the compound of Formula I (or a pharmaceutically acceptable salt thereof) and the 5 mg, 10 mg, 15 mg, or 10 mg of rosuvastatin (or a pharmaceutically acceptable salt thereof, such as, e.g., rosuvastatin calcium) are in separate compositions.

In other specific embodiments, the invention provides a pharmaceutical composition comprising 100 mg of a compound of Formula I or a pharmaceutically acceptable salt thereof and 1.0 mg, 2.0 mg, or 4.0 mg of pitavastatin (or a pharmaceutically acceptable salt thereof, such as pitavastatin calcium). In an alternate embodiment, the 100 mg of the compound of Formula I (or a pharmaceutically acceptable salt thereof) and the 1.0 mg, 2.0 mg, or 4.0 mg of pitavastatin (or a pharmaceutically acceptable salt thereof, such as pitavastatin calcium) are in separate compositions. In certain embodiments, the pharmaceutical composition comprises 200 mg of a compound of Formula I (or a pharmaceutically acceptable salt thereof) and 1.0 mg, 2.0 mg, or 4.0 mg of pitavastatin (or a pharmaceutically acceptable salt thereof, such as, e.g., a calcium salt). In an alternate embodiment, the 200 mg of the compound of Formula I (or a pharmaceutically acceptable salt thereof) and the 1.0 mg, 2.0 mg, or 4.0 mg of pitavastatin (or a pharmaceutically acceptable salt thereof, such as pitavastatin calcium) are in separate compositions.

In some embodiments the pharmaceutical composition comprises 5 mg rosuvastatin or a pharmaceutically acceptable salt thereof, such as, e.g., rosuvastatin calcium, and 100 mg of a compound of Formula I or a pharmaceutically acceptable salt thereof.

In some embodiments the pharmaceutical composition comprises 5 mg rosuvastatin or a pharmaceutically acceptable salt thereof, such as, e.g., rosuvastatin calcium, and 200 mg of a compound of Formula I or a pharmaceutically acceptable salt thereof.

In some embodiments the pharmaceutical composition comprises 10 mg rosuvastatin, such as, e.g., rosuvastatin calcium, or a pharmaceutically acceptable salt thereof and 100 mg of a compound of Formula I or a pharmaceutically acceptable salt thereof.

In some embodiments the pharmaceutical composition comprises 10 mg rosuvastatin or a pharmaceutically acceptable salt thereof, such as, e.g., rosuvastatin calcium, and 200 mg of a compound of Formula I or a pharmaceutically acceptable salt thereof.

In some embodiments the pharmaceutical composition comprises 15 mg rosuvastatin or a pharmaceutically acceptable salt thereof, such as, e.g., rosuvastatin calcium, and 100 mg of a compound of Formula I or a pharmaceutically acceptable salt thereof.

In some embodiments the pharmaceutical composition comprises 15 mg rosuvastatin or a pharmaceutically acceptable salt thereof, such as, e.g., rosuvastatin calcium, and 200 mg of a compound of Formula I or a pharmaceutically acceptable salt thereof.

In some embodiments the pharmaceutical composition comprises 20 mg rosuvastatin or a pharmaceutically acceptable salt thereof, such as, e.g., rosuvastatin calcium, and 100 mg of a compound of Formula I or a pharmaceutically acceptable salt thereof.

In some embodiments the pharmaceutical composition comprises 20 mg rosuvastatin or a pharmaceutically acceptable salt thereof, such as, e.g., rosuvastatin calcium, and 200 mg of a compound of Formula I or a pharmaceutically acceptable salt thereof.

In some embodiments the pharmaceutical composition comprises 20 mg rosuvastatin or a pharmaceutically acceptable salt thereof, such as, e.g., rosuvastatin calcium, and 300 mg of a compound of Formula I or a pharmaceutically acceptable salt thereof.

In some embodiments the pharmaceutical composition comprises 1 mg pitavastatin or a pharmaceutically acceptable salt thereof, such as pitavastatin calcium, and 100 mg of a compound of Formula I or a pharmaceutically acceptable salt thereof.

In some embodiments the pharmaceutical composition comprises 1 mg pitavastatin or a pharmaceutically acceptable salt thereof, such as pitavastatin calcium, and 200 mg of a compound of Formula I or a pharmaceutically acceptable salt thereof.

In some embodiments the pharmaceutical composition comprises 2 mg pitavastatin, or a pharmaceutically acceptable salt thereof, such as pitavastatin calcium, and 100 mg of a compound of Formula I or a pharmaceutically acceptable salt thereof.

In some embodiments the pharmaceutical composition comprises 2 mg pitavastatin or a pharmaceutically acceptable salt thereof, such as pitavastatin calcium, and 200 mg of a compound of Formula I or a pharmaceutically acceptable salt thereof.

In some embodiments the pharmaceutical composition comprises 4 mg pitavastatin or a pharmaceutically acceptable salt thereof, such as pitavastatin calcium, and 100 mg of a compound of Formula I or a pharmaceutically acceptable salt thereof.

In some embodiments the pharmaceutical composition comprises 4 mg pitavastatin or a pharmaceutically acceptable salt thereof, such as pitavastatin calcium, and 200 mg of a compound of Formula I or a pharmaceutically acceptable salt thereof.

In some embodiments the pharmaceutical composition comprises 4 mg pitavastatin or a pharmaceutically acceptable salt thereof, such as pitavastatin calcium, and 300 mg of a compound of Formula I or a pharmaceutically acceptable salt thereof.

In some embodiments, any of the compositions or combinations of compositions of the invention set forth above may be used to treat or prevent atherosclerosis or other cholesterol- or lipid-related disease or disorder as described for any of the methods of the invention set forth below.

### Methods of Treatment

The invention provides methods of treating and/or preventing atherosclerosis or other cholesterol- or lipid-related disease by co-administering therapeutically effective amounts of a compound of Formula I (or a pharmaceutically acceptable salt thereof) and rosuvastatin or pitavastatin (or a pharmaceutically acceptable salt of rosuvastatin or pitavastatin, such as, e.g., a calcium salt) as described herein. In some embodiments, the methods of the invention include methods of reducing atherosclerosis, methods of inhibiting or delaying the progression of atherosclerosis, methods of reducing or preventing MAVEs, methods of reducing percent atheroma volume; and/or methods of reducing total atheroma volume, in a patient by co-administering a compound of Formula I (or a pharmaceutically acceptable salt thereof, and rosuvastatin or pitavastatin (or a pharmaceutically acceptable salt of rosuvastatin or pitavastatin, such as, e.g., a calcium salt). In certain embodiments, co-administration of a compound of Formula I (or a pharmaceutically acceptable salt thereof) and rosuvastatin or pitavastatin (or a pharmaceutically acceptable salt of rosuvastatin or pitavastatin, such as, e.g., a calcium salt) can be administered to stop progression of atherosclerosis more effectively than administration of rosuvastatin or rosuvastatin calcium, or pitavastatin or pitavastatin calcium alone. In some embodiments, co-administration of a compound of Formula I (or a pharmaceutically acceptable salt thereof) and rosuvastatin or pitavastatin (or a pharmaceutically acceptable salt of rosuvastatin or pitavastatin, such as, e.g., a calcium salt) results in regression of atherosclerosis in a patient.

In some embodiments, the disorders that may be treated or prevented with the compositions and methods of the invention include cardiovascular diseases, peripheral vascular diseases, renal bed vascular diseases, and cerebrovascular diseases. In some embodiments, the disorders that may be treated or prevented with the compositions and methods of the invention include metabolic diseases related to coronary atherosclerosis and the build-up of arterial plaque.

In some embodiments, the methods of the invention comprise administration of a compound of Formula I twice a day and administration of rosuvastatin or pitavastatin (or a pharmaceutically acceptable salt of rosuvastatin or pitavastatin, such as, e.g., a calcium salt) once a day.

In certain embodiments, the methods of the invention comprise administering a compound of Formula I (or a pharmaceutically acceptable salt thereof) and 5 mg of rosuvastatin (or a pharmaceutically acceptable salt thereof, e.g., rosuvastatin calcium) in a single formulation once or twice per day. In alternate embodiments, the compound of Formula I (or a pharmaceutically acceptable salt thereof) and 5 mg of rosuvastatin (or a pharmaceutically acceptable salt thereof, e.g., rosuvastatin calcium) are administered separately in the methods of the invention. In such embodiments, the compound of Formula I (or a pharmaceutically acceptable salt thereof) and 5 mg of rosuvastatin (or a pharmaceutically acceptable salt thereof, e.g., rosuvastatin calcium) may be administered simultaneously or sequentially.

In some embodiments of the methods of the invention, the compound of Formula I (or a pharmaceutically acceptable salt thereof) and 1.0 mg, 2.0 mg, or 4.0 mg of pitavastatin (or a pharmaceutically acceptable salt thereof, such as pitavastatin calcium) are administered in a single formulation once or twice per day. In alternate embodiments, the compound of Formula I (or a pharmaceutically acceptable salt thereof) and 0.1.0 mg, 2.0 mg, or 4.0 mg of pitavastatin (or a pharmaceutically acceptable salt thereof, such as pitavastatin calcium) are administered separately in the methods of the invention. In such embodiments, the compound of Formula I (or a pharmaceutically acceptable salt) thereof and 1.0 mg, 2.0 mg, or 4.0 mg of pitavastatin (or a pharmaceutically acceptable salt thereof, such as pitavastatin calcium) may be administered simultaneously or sequentially.

In some embodiments, the methods of the invention comprise administering a single composition comprising 100, 150, 200, 250, or 300 mg of the compound of Formula I (or a pharmaceutically acceptable salt thereof) and 5, 10, 15, 20 mg of rosuvastatin (or a pharmaceutically acceptable salt thereof, e.g., rosuvastatin calcium) once daily or twice daily. In some embodiments, the methods of the invention comprise administration of separate compositions, one comprising 100, 150, 200, 250, or 300 mg of the compound of Formula I (or a pharmaceutically acceptable salt thereof) and one comprising 5, 10, 15, or 20 mg of rosuvastatin (or a pharmaceutically acceptable salt thereof, e.g., rosuvastatin calcium). In more specific embodiments, the methods of the invention comprise administering 200 mg of the compound of Formula I (or a pharmaceutically acceptable salt thereof) and 5, 10, 15, or 20 mg of rosuvastatin (or a pharmaceutically acceptable salt thereof, e.g., rosuvastatin calcium) as a single composition or as separate compositions once a day to treat atherosclerosis. In an alternate embodiment, the methods of the invention comprise administration of 100 mg of the compound of Formula I (or a pharmaceutically acceptable salt thereof) twice each day and administration of 5, 10, 15, or 20 mg of rosuvastatin (or a pharmaceutically acceptable salt thereof, e.g., rosuvastatin calcium) once a day.

In some embodiments of the methods of the invention, a single composition comprising 100, 150, 200, 250, or 300 mg of the compound of Formula I (or a pharmaceutically acceptable salt thereof, such as pitavastatin calcium) and 1.0 mg, 2.0 mg, or 4.0 mg of pitavastatin (or a pharmaceutically acceptable salt thereof, such as pitavastatin calcium) is administered once daily or twice daily. In some embodiments of the methods of the invention, separate compositions, one comprising 100, 150, 200, 250, or 300 mg of the compound of Formula I (or a pharmaceutically acceptable salt thereof) and one comprising 1.0 mg, 2.0 mg, or 4.0 mg of pitavastatin (or a pharmaceutically acceptable salt thereof, such as pitavastatin calcium) are administered. In more specific embodiments of the methods of the invention, 200 mg of the compound of Formula I (or a pharmaceutically acceptable salt thereof) and 1.0 mg, 2.0 mg, or 4.0 mg of pitavastatin (or a pharmaceutically acceptable salt thereof, such as pitavastatin calcium) are administered as a single composition or as separate compositions once a day to treat atherosclerosis. In an alternate embodiment, the method comprises administration of 100 mg of the compound of Formula I (or a pharmaceutically acceptable salt thereof) twice each day and administration of 1.0 mg, 2.0 mg, or 4.0 mg of pitavastatin (or a pharmaceutically acceptable salt thereof, such as pitavastatin calcium) once a day.

In some embodiments of the methods of the invention, the therapeutically effective amount of rosuvastatin or a pharmaceutically acceptable salt thereof, such as, e.g., rosuvastatin calcium, is 5 mg and the therapeutically effective amount of a compound of Formula I or a pharmaceutically acceptable salt thereof is 100 mg. In some embodiments the rosuvastatin or a pharmaceutically acceptable salt thereof, such as, e.g., rosuvastatin calcium, and the compound of Formula I or a pharmaceutically acceptable salt thereof are administered as a single composition. In some embodiments the rosuvastatin or a pharmaceutically acceptable salt thereof, such as, e.g., rosuvastatin calcium, and the compound of Formula I or a pharmaceutically acceptable salt thereof are administered as separate compositions.

In some embodiments of the methods of the invention, the therapeutically effective amount of rosuvastatin or a pharmaceutically acceptable salt thereof, such as, e.g., rosuvastatin calcium, is 5 mg and the therapeutically effective amount of a compound of Formula I or a pharmaceutically acceptable salt thereof is 200 mg. In some embodiments the rosuvastatin or a pharmaceutically acceptable salt thereof, such as, e.g., rosuvastatin calcium, and the compound of Formula I or a pharmaceutically acceptable salt thereof are administered as a single composition. In some embodiments the rosuvastatin or a pharmaceutically acceptable salt thereof, such as, e.g., rosuvastatin calcium, and the compound of Formula I or a pharmaceutically acceptable salt thereof are administered as separate compositions.

In some embodiments of the methods of the invention, the therapeutically effective amount of rosuvastatin or a pharmaceutically acceptable salt thereof, such as, e.g., rosuvastatin calcium, is 10 mg and the therapeutically effective amount of a compound of Formula I or a pharmaceutically acceptable salt thereof is 100 mg. In some embodiments the rosuvastatin or a pharmaceutically acceptable salt thereof, such as, e.g., rosuvastatin calcium, and the compound of Formula I or a pharmaceutically acceptable salt thereof are administered as a single composition. In some embodiments the rosuvastatin or a pharmaceutically acceptable salt thereof, such as, e.g., rosuvastatin calcium, and the compound of Formula I or a pharmaceutically acceptable salt thereof are administered as separate compositions.

In some embodiments of the methods of the invention, the therapeutically effective amount of rosuvastatin or a pharmaceutically acceptable salt thereof, such as, e.g., rosuvastatin calcium, is 10 mg and the therapeutically effective amount of a compound of Formula I or a pharmaceutically acceptable salt thereof is 200 mg. In some embodiments, the rosuvastatin or a pharmaceutically acceptable salt thereof, such as, e.g., rosuvastatin calcium, and the compound or Formula I or a pharmaceutically acceptable salt thereof are administered as a single composition. In some embodiments, the rosuvastatin or a pharmaceutically acceptable salt thereof, such as, e.g., rosuvastatin calcium, and the compound of Formula I or a pharmaceutically acceptable salt thereof are administered as separate compositions.

In some embodiments of the methods of the invention, the therapeutically effective amount of rosuvastatin or a pharmaceutically acceptable salt thereof, such as, e.g., rosuvastatin calcium, is 15 mg and the therapeutically effective amount of a compound of Formula I or a pharmaceutically acceptable salt thereof is 200 mg. In some embodiments, the rosuvastatin or a pharmaceutically acceptable salt thereof, such as, e.g., rosuvastatin calcium, and the compound of Formula I or a pharmaceutically acceptable salt thereof are administered as a single composition. In some embodiments, the rosuvastatin or a pharmaceutically acceptable salt thereof, such as, e.g., rosuvastatin calcium, and the compound of Formula I or a pharmaceutically acceptable salt thereof are administered as separate compositions.

In some embodiments of the methods of the invention, the therapeutically effective amount of rosuvastatin or a pharmaceutically acceptable salt thereof, such as, e.g., rosuvastatin calcium, is 20 mg and the therapeutically effective amount of a compound of Formula I or a pharmaceutically acceptable salt thereof is 200 mg.

In some embodiments of the methods of the invention, the therapeutically effective amount of rosuvastatin or a pharmaceutically acceptable salt thereof, such as, e.g., rosuvastatin calcium, is 20 mg and the therapeutically effective amount of a compound of Formula I or a pharmaceutically acceptable salt thereof is 300 mg.

In some embodiments, including each embodiment recited above, the methods of the invention comprise administering the rosuvastatin or a pharmaceutically acceptable salt thereof, such as, e.g., rosuvastatin calcium, and the compound of Formula I or a pharmaceutically acceptable salt thereof as a single composition. In some embodiments, including any of those recited above, the rosuvastatin or a pharmaceutically acceptable salt thereof, such as, e.g., rosuvastatin calcium, and the compound of Formula I or a pharmaceutically acceptable salt thereof are administered as separate compositions.

In some embodiments of the methods of the invention, the therapeutically effective amount of pitavastatin or a pharmaceutically acceptable salt thereof, such as pitavastatin calcium, is 1 mg and the therapeutically effective amount of a compound of Formula I or a pharmaceutically acceptable salt thereof is 100 mg. In some embodiments the pitavastatin or a pharmaceutically acceptable salt thereof, such as pitavastatin calcium, and the compound of Formula I or a pharmaceutically acceptable salt thereof are administered as a single composition. In some embodiments the pitavastatin or a pharmaceutically acceptable salt thereof, such as, e.g., a calcium salt, and the compound of Formula I or a pharmaceutically acceptable salt thereof are administered as separate compositions.

In some embodiments of the methods of the invention, the therapeutically effective amount of pitavastatin or a pharmaceutically acceptable salt thereof, such as pitavastatin calcium, is 1 mg and the therapeutically effective amount of a compound of Formula I or a pharmaceutically acceptable salt thereof is 200 mg. In some embodiments the pitavastatin or a pharmaceutically acceptable salt thereof, such as pitavastatin calcium, and the compound of Formula I or a pharmaceutically acceptable salt thereof are administered as a single composition. In some embodiments the pitavastatin or a pharmaceutically acceptable salt thereof, such as pitavastatin calcium, and the compound of Formula I or a pharmaceutically acceptable salt thereof are administered as separate compositions.

In some embodiments of the methods of the invention, the therapeutically effective amount of pitavastatin or a pharmaceutically acceptable salt thereof, such as pitavastatin calcium, is 2 mg and the therapeutically effective amount of a compound of Formula I or a pharmaceutically acceptable salt thereof is 100 mg. In some embodiments the pitavastatin or a pharmaceutically acceptable salt thereof, such as pitavastatin calcium, and the compound of Formula I or a pharmaceutically acceptable salt thereof are administered as a single composition. In some embodiments the pitavastatin or a pharmaceutically acceptable salt thereof, such as pitavastatin calcium, and the compound of Formula I or a pharmaceutically acceptable salt thereof are administered as separate compositions.

In some embodiments of the methods of the invention, the therapeutically effective amount of pitavastatin or a pharmaceutically acceptable salt thereof, such as pitavastatin calcium, is 2 mg and the therapeutically effective amount of a compound of Formula I or a pharmaceutically acceptable salt thereof is 200 mg. In some embodiments, the pitavastatin or a pharmaceutically acceptable salt thereof, such as pitavastatin calcium, and the compound of Formula I or a pharmaceutically acceptable salt thereof are administered as a single composition. In some embodiments, the pitavastatin or a pharmaceutically acceptable salt thereof, such as pitavastatin calcium, and the compound of Formula I or a pharmaceutically acceptable salt thereof are administered as separate compositions.

In some embodiments of the methods of the invention, the therapeutically effective amount of pitavastatin or a pharmaceutically acceptable salt thereof, such as pitavastatin calcium, is 4 mg and the therapeutically effective amount of a compound of Formula I or a pharmaceutically acceptable salt thereof is 100 mg. In some embodiments, the pitavastatin or a pharmaceutically acceptable salt thereof, such as pitavastatin calcium, and the compound of Formula I or a pharmaceutically acceptable salt thereof are administered as a single composition. In some embodiments, the pitavastatin or a pharmaceutically acceptable salt thereof, such as pitavastatin calcium, and the compound of Formula I or a pharmaceutically acceptable salt thereof are administered as separate compositions.

In some embodiments of the methods of the invention, the therapeutically effective amount of pitavastatin or a pharmaceutically acceptable salt thereof, such as pitavastatin calcium, is 4 mg and the therapeutically effective amount of a compound of Formula I or a pharmaceutically acceptable salt thereof is 200 mg.

In some embodiments of the methods of the invention, the therapeutically effective amount of pitavastatin or a pharmaceutically acceptable salt thereof, such as pitavastatin calcium, is 4 mg and the therapeutically effective amount of a compound of Formula I or a pharmaceutically acceptable salt thereof is 300 mg.

In some embodiments, including each embodiment recited above, the methods of the invention comprise administering the pitavastatin or a pharmaceutically acceptable salt thereof, such as pitavastatin calcium, and the compound of Formula I or a pharmaceutically acceptable salt thereof as a single composition. In some embodiments, including any of those recited above, the pitavastatin or a pharmaceutically acceptable salt thereof, such as pitavastatin calcium, and the compound of Formula I or a pharmaceutically acceptable salt thereof are administered as separate compositions.

### EXAMPLES

### Example 1

To demonstrate the ability of the compounds of Formula I to upregulate ApoA-I, mRNA encoding ApoA-I was quantitated in tissue culture cells to measure the transcriptional up-regulation of ApoA-I when treated with a compound of Formula I. HepG2 cells (˜2×10⁵ per well) were placed in a 24-well plate in ˜400 μL MEM, supplemented with 0.5% (v/v) FBS, 24 h before addition of the compound of interest. At time of harvesting, the spent media was removed from the HepG2 cells and immediately placed on ice (for immediate use) or at −80° C. (for future use) in ApoA-I and albumin ELISAs. The cells remaining in the plate wells were rinsed in 200 μL PBS. PBS was carefully removed to avoid removing any loosely attached cells.

Once the PBS was removed, 85 μL cell lysis solution was added to the cells in each well and incubated for 5-10 min at room temperature, to allow for complete cell lysis and detachment. mRNA was then prepared using the “mRNA Catcher PLUS plate” from Invitrogen, according to the protocol supplied. After the last wash, as much wash buffer as possible was aspirated without allowing the wells to dry. Elution Buffer (E3, 80 μL) was then added to each well. mRNA was then eluted by incubating the mRNA Catcher PLUS plate with Elution Buffer for 5 min at 68° C. and then immediately placing the plate on ice.

The eluted mRNA was isolated and then used in a one-step real-time room temperature-PCR reaction, using components of the Ultra Sense Kit together with Applied Biosystems primer-probe mixes. Real-time PCR data was analyzed, using the Ct values, to determine the fold induction of each unknown sample, relative to the control (that is, relative to the control for each independent DMSO concentration).

The following compounds of Formula I were evaluated in this assay. An active compound is one that causes a >15% increase in ApoA-I mRNA at a concentration less than or equal to 100 uM.

| | Effect |
|---|---|
| | on |
| | ApoA-I |
| | mRNA |
| Compound Name | levels |
| N-(2-(4-(5,7-Dimethoxy-4-oxo-3,4-dihydroquinazolin-2-yl)- | Active |
| 2,6-dimethylphenoxy)ethyl)-4-methylbenzamide | |
| 2-(4-(5,7-Dimethoxy-4-oxo-3,4-dihydroquinazolin-2-yl)-2,6- | Active |
| dimethyl-phenoxy)ethyl methylcarbamate | |
| 2-(4-(5,7-Dimethoxy-4-oxo-3,4-dihydroquinazolin-2-yl)-2,6- | Active |
| dimethyl-phenoxy)ethyl propylcarbamate | |
| N-(2-(4-(5,7-dimethoxy-4-oxo-3,4-dihydroquinazolin-2-yl)- | Active |
| 2,6-dimethylphenoxy)ethyl)methanesulfonamide | |
| 4-chloro-N-(2-(4-(5,7-dimethoxy-4-oxo-3,4- | Active |
| dihydroquinazolin-2-yl)-2,6- | |
| dimethylphenoxy)ethyl)benzenesulfonamide | |
| N-(2-(4-(5,7-dimethoxy-4-oxo-3,4-dihydroquinazolin-2-yl)- | Active |
| 2,6-dimethylphenoxy)ethyl)-4-methoxybenzenesulfonamide | |
| 2-(4-(2-aminoethoxy)-3,5-dimethylphenyl)-5,7- | Active |
| dimethoxyquinazolin-4(3H)-one | |
| N1-(2-(4-(5,7-dimethoxy-4-oxo-3,4-dihydroquinazolin-2-yl)- | Active |
| 2,6-dimethylphenoxy)ethyl)-N2-methylphthalamide | |
| 2-(2-(4-(5,7-dimethoxy-4-oxo-3,4-dihydroquinazolin-2-yl)- | Inactive |
| 2,6-dimethylphenoxy)ethyl)isoindoline-1,3-dione | |
| 2-(4-hydroxy-3-methylphenyl)-5,7-dimethoxyquinazolin- | Active |
| 4(3H)-one | |
| 2-(4-(2-hydroxyethoxy)-3-methylphenyl)-5,7- | Active |
| dimethoxyquinazolin-4(3H)-one | |
| 2-(4-(benzyloxy)-3,5-dimethylphenyl)-5,7- | Active |
| dimethoxyquinazolin-4(3H)-one | |
| 6-bromo-2-(4-(2-(tert-butyldimethylsilyloxy)ethoxy)-3,5- | Active |
| dimethylphenyl)quinazolin-4(3H)-one | |
| 6-bromo-2-(4-(2-hydroxyethoxy)-3,5- | Active |
| dimethylphenyl)quinazolin-4(3H)-one | |
| 6-bromo-2-(4-hydroxy-3,5-dimethylphenyl)quinazolin-4(3H)- | Active |
| one | |
| (E)-N′-(4-(5,7-dimethoxy-4-oxo-3,4-dihydroquinazolin-2-yl)- | Active |
| 2,6-dimethylphenyl)-N,N-dimethylformimidamide | |
| 2-(3,5-dimethylphenyl)-5,7-dimethoxyquinazolin-4(3H)-one | Active |
| 5,7-dimethoxy-2-(3-nitrophenyl)quinazolin-4(3H)-one | Inactive |
| 2-(4-amino-3,5-dimethylphenyl)-5,7-dimethoxyquinazolin- | Active |
| 4(3H)-one | |
| 5,7-dimethoxy-2-(4-methoxy-3,5-dimethylphenyl)quinazolin- | Active |
| 4(3H)-one | |
| 2-(2-chloro-6-methylpyridin-4-yl)-5,7-dimethoxyquinazolin- | Active |
| 4(3H)-one | |
| 5,7-dimethoxy-2-(6-(4-(methylthio)phenyl)pyridin-2- | Active |
| yl)quinazolin-4(3H)-one | |
| 5,7-dimethoxy-2-(6-methylpyridin-2-yl)quinazolin-4(3H)-one | Active |
| 5,7-dimethoxy-2-(6-(4-(methylsulfonyl)phenyl)pyridin-2- | Active |
| yl)quinazolin-4(3H)-one | |
| 5,7-dimethoxy-2-o-tolylquinazolin-4(3H)-one | Active |
| 2-(4-(2-hydroxyethoxy)-3,5-dimethylphenyl)-5- | Active |
| methoxyquinazolin-4(3H)-one | |
| 2-(2-chlorophenyl)-5,7-dimethoxyquinazolin-4(3H)-one | Active |
| 2-(4-(2-hydroxyethoxy)-3,5-dimethylphenyl)-6- | Active |
| methoxyquinazolin-4(3H)-one | |
| 4-(4-ethoxy-5,7-dimethoxyquinazolin-2-yl)-2,6- | Inactive |
| dimethylphenol | |
| 2-(4-hydroxy-3,5-dimethylphenyl)-5,7-dimethoxy-6- | Active |
| (morpholinomethyl)quinazolin-4(3H)-one | |
| 5,7-dichloro-2-(4-(2-hydroxyethoxy)-3,5- | Active |
| dimethylphenyl)quinazolin-4(3H)-one | |
| 5,7-dimethoxy-2-(4-(2-methoxyethoxy)-3,5- | Active |
| dimethylphenyl)quinazolin-4(3H)-one | |
| 2-(4-(bis(2-hydroxyethyl)amino)phenyl)-5,7- | Active |
| dimethoxypyrido[2,3-d]pyrimidin-4(3H)-one | |
| 2-(4-(2-hydroxyethoxy)-3,5-dimethylphenyl)-5,7- | Active |
| dimethylquinazolin-4(3H)-one | |
| 2-(4-(5,7-dimethoxyquinazolin-2-yl)-2,6- | Active |
| dimethylphenoxy)ethanol | |
| 2-(4-hydroxy-3-(2-hydroxyethyl)phenyl)-5,7- | Active |
| dimethoxyquinazolin-4(3H)-one | |
| 5-hydroxy-2-(4-hydroxy-3,5-dimethylphenyl)-7- | Active |
| methoxyquinazolin-4(3H)-one | |
| 7-(4-hydroxy-3,5-dimethylphenyl)-2,4-diisopropoxy-1,6- | Active |
| naphthyridin-5(6H)-one | |
| 2-(4-(2-(benzyloxy)ethoxy)-3,5-dimethylphenyl)-5,7- | Inactive |
| dimethoxypyrido[2,3-d]pyrimidin-4(3H)-one | |
| N-(2-(4-(2-hydroxyethoxy)-3,5-dimethylphenyl)-4-oxo-3,4- | Active |
| dihydroquinazolin-6-yl)acetamide | |
| 2-(3,5-dimethyl-4-(2-(pyrrolidin-1-yl)ethoxy)phenyl)-5,7- | Active |
| dimethoxyquinazolin-4(3H)-one | |
| 2-(4-hydroxy-3,5-dimethylphenyl)-5,7-dimethoxy-1- | Active |
| methylquinazolin-4(1H)-one | |
| 2-(4-(2-hydroxyethoxy)-3,5-dimethylphenyl)-5,7- | Active |
| dimethoxypyrido[2,3-d]pyrimidin-4(3H)-one | |
| 5,7-dimethoxy-2-(4-methoxy-3- | Active |
| (morpholinomethyl)phenyl)quinazolin-4(3H)-one | |
| 2-(4-hydroxy-3,5-dimethylphenyl)-5,7-dimethoxypyrido[2,3- | Active |
| d]pyrimidin-4(3H)-one | |
| 2-(4-((4-ethylpiperazin-1-yl)methyl)phenyl)-5,7- | Active |
| dimethoxyquinazolin-4(3H)-one | |
| 5,7-dimethoxy-2-(4-(morpholinomethyl)phenyl)quinazolin- | Active |
| 4(3H)-one | |
| N-(4-(5,7-dimethoxy-4-oxo-3,4-dihydroquinazolin-2- | Active |
| yl)phenyl)-2-hydroxyacetamide | |
| 2-(4-(5,7-dimethoxy-4-oxo-3,4-dihydroquinazolin-2-yl)-2,6- | Active |
| dimethylphenoxy)acetic acid | |
| 2,4-dimethoxy-7-(4-methoxy-3,5-dimethylphenyl)-1,6- | Active |
| naphthyridin-5(6H)-one | |
| 2-(4-hydroxy-3,5-dimethylphenyl)-6- | Active |
| (morpholinomethyl)quinazolin-4(3H)-one | |
| 7-(4-(2-hydroxyethoxy)-3,5-dimethylphenyl)-2,4-dimethoxy- | Active |
| 1,6-naphthyridin-5(6H)-one | |
| N-(4-(5,7-dimethoxy-4-oxo-3,4-dihydroquinazolin-2-yl)-2,6- | Active |
| dimethylphenyl)-2-hydroxyacetamide | |
| 5,7-dimethoxy-2-(4-((4-methylpiperazin-1- | Active |
| yl)methyl)phenyl)quinazolin-4(3H)-one | |
| 2-(4-(2-hydroxyethoxy)-3,5-dimethylphenyl)-6,7- | Active |
| dimethoxyquinazolin-4(3H)-one | |
| 2-(4-(5,7-dimethoxy-4-oxo-3,4-dihydroquinazolin-2-yl)-2- | Active |
| methoxyphenoxy)acetic acid | |
| 2-(4-(2-hydroxyethoxy)-3-methoxyphenyl)-5,7- | Active |
| dimethoxyquinazolin-4(3H)-one | |
| 2-(3-chloro-4-(2-hydroxyethoxy)phenyl)-5,7- | Active |
| dimethoxyquinazolin-4(3H)-one | |
| 2-(4-(6,7-dimethoxy-4-oxo-3,4-dihydroquinazolin-2-yl)-2,6- | Active |
| dimethylphenoxy)acetamide | |
| N-(2-(4-hydroxy-3,5-dimethylphenyl)-4-oxo-3,4- | Active |
| dihydroquinazolin-6-yl)acetamide | |
| 3-(4-(5,7-dimethoxy-4-oxo-3,4-dihydroquinazolin-2- | Active |
| yl)phenyl)propanoic acid | |
| 2-(2-(4-chlorophenoxy)pyridin-3-yl)-5,7- | Inactive |
| dimethoxyquinazolin-4(3H)-one | |
| 3-(4-hydroxy-3,5-dimethylphenyl)-6,8-dimethoxy-7- | Active |
| (morpholinomethyl)isoquinolin-1(2H)-one | |
| 7-(3,5-dimethyl-4-(2-morpholinoethoxy)phenyl)-2,4- | Active |
| dimethoxy-1,6-naphthyridin-5(6H)-one | |
| 5,7-dimethoxy-2-(4-morpholinophenyl)quinazolin-4(3H)-one | Active |
| 2-(2,3-dihydrobenzo[b][1,4]dioxin-6-yl)-6,7- | Active |
| dimethoxyquinazolin-4(3H)-one | |
| 2-(4-(bis(2-hydroxyethyl)amino)phenyl)-6,7- | Active |
| dimethoxyquinazolin-4(3H)-one | |
| 2-(4-(bis(2-hydroxyethyl)amino)phenyl)-5,7- | Active |
| dimethoxyquinazolin-4(3H)-one | |
| 2-(4-(bis(2-hydroxyethyl)amino)phenyl)quinazolin-4(3H)-one | Active |
| 2-(4-(dimethylamino)pyridin-1-yl)-6,7-dimethoxyquinazolin- | Active |
| 4(3H)-one | |
| 5,7-dimethoxy-2-(4-(4-methylpiperazin-1- | Active |
| yl)phenyl)quinazolin-4(3H)-one | |
| 2-(2,3-dihydrobenzo[b][1,4]dioxin-6-yl)-5,7- | Active |
| dimethoxyquinazolin-4(3H)-one | |
| 2-(4-(4-oxo-3,4-dihydroquinazolin-2-yl)phenoxy)acetamide | Active |
| 2-(4-(dimethylamino)naphthalene-1-yl)quinazolin-4(3H)-one | Active |
| 2-(4-(4-oxo-3,4-dihydroquinazolin-2-yl)phenoxy)acetic acid | Active |
| 2-(4-(5,7-dimethoxy-4-oxo-3,4-dihydroquinazolin-2- | Active |
| yl)phenoxy)acetamide | |
| 2-(4-(dimethylamino)naphthalene-1-yl)-5,7- | Active |
| dimethoxyquinazolin-4(3H)-one | |
| 2-(2,3-dihydrobenzo[b][1,4]dioxin-6-yl)quinazolin-4(3H)-one | Active |
| 2-(4-(2-hydroxyethoxy)-3,5-dimethylphenyl)quinazolin- | Active |
| 4(3H)-one | |
| 2-(3,5-dimethyl-4-(2-morpholinoethoxy)phenyl)quinazolin- | Active |
| 4(3H)-one | |
| 5,7-dimethoxy-2-(pyridin-4-yl)quinazolin-4(3H)-one | Active |
| 2-(3-chloro-4-hydroxyphenyl)-5,7-dimethoxyquinazolin- | Active |
| 4(3H)-one | |
| 2-(4-hydroxy-3-methoxyphenyl)-5,7-dimethoxyquinazolin- | Active |
| 4(3H)-one | |
| 5,7-dimethoxy-2-(4-methoxyphenyl)quinazolin-4(3H)-one | Active |
| 2-(3,5-dimethoxyphenyl)-5,7-dimethoxyquinazolin-4(3H)-one | Active |
| 2-(3,5-di-tert-butyl-4-hydroxyphenyl)-5,7- | Active |
| dimethoxyquinazolin-4(3H)-one | |
| 5,7-dimethoxy-2-(pyridin-2-yl)quinazolin-4(3H)-one | Active |
| 2-(4-hydroxyphenyl)-5,7-dimethoxyquinazolin-4(3H)-one | Active |
| 3-(3,5-dimethyl-4-(2-(4-methylpiperazin-1-yl)ethoxy)phenyl)- | Active |
| 6,8-dimethoxyisoquinolin-1(2H)-one | |
| 4-(2-(4-(6,8-dimethoxyisoquinolin-3-yl)-2,6- | Active |
| dimethylphenoxy)ethyl)morpholine | |
| 2-(3,5-dimethyl-4-(2-morpholinoethoxy)phenyl)-5,7- | Active |
| dimethoxyquinazolin-4(3H)-one | |
| 5,7-dimethoxy-2-p-tolylquinazolin-4(3H)-one | Inactive |
| 2-(4-(2-hydroxyethoxy)-3,5-dimethylphenyl)-5,7- | Active |
| dimethoxyquinazolin-4(3H)-one | |
| 3-(3,5-dimethyl-4-(2-morpholinoethoxy)phenyl)-6,8- | Active |
| dimethoxyisoquinolin-1(2H)-one | |
| 4-(6,8-dimethoxyisoquinolin-3-yl)-2,6-dimethylphenol | Active |
| 3-(4-hydroxy-3,5-dimethylphenyl)-6,8-dimethoxy-2-methyl- | Active |
| 7-(morpholinomethyl) isoquinolin-1(2H)-one | |
| 3-(4-hydroxy-3,5-dimethylphenyl)-6,8-dimethoxy-2,7- | Active |
| dimethylisoquinolin-1(2H)-one | |
| 7-(4-hydroxy-3,5-dimethylphenyl)-2,4-dimethoxy-1,6- | Active |
| naphthyridin-5(6H)-one | |
| 6,8-dimethoxy-3-(4-hydroxy-3,5-dimethylphenyl)-2H-1,2- | Active |
| benzothiazine-1,1-dioxide | |
| 3-(4-(2-hydroxy-2-methylpropoxy)-3,5-dimethylphenyl)-6,8- | Active |
| dimethoxyisoquinolin-1(2H)-one | |
| 2-(4-hydroxy-3,5-dimethylphenyl)-5,7-dimethoxyquinazolin- | Active |
| 4(3H)-one | |
| 2-hydroxy-7-(4-hydroxy-3,5-dimethylphenyl)-4-methoxy-1,6- | Active |
| naphthyridin-5(6H)-one | |
| 3-(4-hydroxy-3,5-dimethylphenyl)-7- | Active |
| (morpholinomethyl)isoquinolin-1(2H)-one | |
| 3-(4-(2-(dimethylamino)ethoxy)-3,5-dimethylphenyl)-6,8- | Active |
| dimethoxyisoquinolin-1(2H)-one | |
| 3-(4-(2-hydroxyethoxy)-3,5-dimethylphenyl)-6,8- | Active |
| dimethoxyisoquinolin-1(2H)-one | |
| 7-(4-hydroxy-3,5-dimethylphenyl)-1,6-naphthyridin-5(6H)- | Active |
| one | |
| 3-(4-hydroxy-3,5-dimethylphenyl)-6,8-dimethoxyisoquinolin- | Active |
| 1(2H)-one | |
| 3-(4-hydroxy-3,5-dimethylphenyl)-6,8-dimethoxy-2- | Active |
| methylisoquinolin-1(2H)-one | |
| 3-(4-hydroxyphenyl)-6,8-dimethoxyisoquinolin-1(2H)-one | Active |
| 3-(3-fluoro-4-hydroxyphenyl)-5-methoxyisoquinolin-1(2H)- | Inactive |
| one | |
| 4-(1,6-naphthyridin-7-yl)phenol | Active |
| 4-(1-Oxo-1,2-dihydroisoquinolin-3-yl)phenyl 2-amino-5- | Active |
| guanidinopentanoate | |
| 4-(Isoquinolin-3-yl)phenyl 2-amino-5-guanidinopentanoate | Active |
| tetrahydrochloride | |
| 4-Isoquinolin-3-yl-phenol | Active |
| 3-(4-Hydroxyphenyl)-2H-isoquinolin-1-one | Active |
| 2-(4-(5,7-dimethoxy-4-oxo-3,4-dihydroquinazolin-2-yl)-2,6- | Active |
| dimethylphenoxy)ethyl cyclohexylcarbamate | |
| N-(2-(4-(5,7-dimethoxy-4-oxo-3,4-dihydroquinazolin-2-yl)- | Active |
| 2,6-dimethylphenoxy)ethyl)benzenesulfonamide | |
| N-(2-(4-(5,7-dimethoxy-4-oxo-3,4-dihydroquinazolin-2-yl)- | Active |
| 2,6-dimethylphenoxy)ethyl)-4-methylbenzenesulfonamide | |
| N-(2-(4-(5,7-dimethoxy-4-oxo-3,4-dihydroquinazolin-2-yl)- | Active |
| 2,6-dimethylphenoxy)ethyl)-4-methoxybenzamide | |
| N-(2-(4-(5,7-dimethoxy-4-oxo-3,4-dihydroquinazolin-2-yl)- | Active |
| 2,6-dimethylphenoxy)ethyl)acetamide | |
| N-(2-(4-(5,7-dimethoxy-4-oxo-3,4-dihydroquinazolin-2-yl)- | Active |
| 2,6-dimethylphenoxy)ethyl)benzamide | |
| N-(2-(4-(5,7-dimethoxy-4-oxo-3,4-dihydroquinazolin-2-yl)- | Active |
| 2,6-dimethylphenoxy)ethyl)isobutyramide | |
| 1-(2-(4-(5,7-dimethoxy-4-oxo-3,4-dihydroquinazolin-2-yl)- | Active |
| 2,6-dimethylphenoxy)ethyl)-3-methylurea | |
| 1-(2-(4-(5,7-dimethoxy-4-oxo-3,4-dihydroquinazolin-2-yl)- | Active |
| 2,6-dimethylphenoxy)ethyl)-3-(4-methoxyphenyl)urea | |
| 1-(2-(4-(5,7-dimethoxy-4-oxo-3,4-dihydroquinazolin-2-yl)- | Active |
| 2,6-dimethylphenoxy)ethyl)-3-phenylurea | |
| 3-(2-(4-(5,7-dimethoxy-4-oxo-3,4-dihydroquinazolin-2-yl)- | Active |
| 2,6-dimethylphenoxy)ethyl)-1,1-dimethylurea | |

### Example 2

The ASSURE I (ApoA-I Synthesis Stimulation and Intravascular Ultrasound for Coronary Atheroma Regression Evaluation) trial was a phase two multi-center, double-blind, randomized, parallel group, placebo-controlled clinical trial for the assessment of coronary plaque changes with RVX-208, as determined by intravascular ultrasound.

It was a 26-week (or 6 month) active-treatment period double-blind, placebo-controlled, two-arm parallel group (allocation ratio 3:1) study of RVX-208 at a daily dose of 200 mg or matching placebo administered daily to patients with a low HDL-c level who required coronary angiography for a clinical indication. In total, 324 patients were enrolled.

The ASSURE I study was designed to characterize the early effects of ApoA-I synthesis with RVX-208 on coronary atherosclerotic disease when administered to patients with coronary artery disease and have a low HDL-c level, as assessed using intravascular ultrasound (IVUS) in addition to standard background therapy, including, but not limited to, statins.

The objectives were to evaluate the effect of RVX-208 on the change in burden of coronary atherosclerosis, as measured by percent atheroma volume (PAV) and total atheroma volume (TAV), in patients with coronary artery disease with a low level of HDL-c and requiring angiography for a clinical indication. The inclusion criteria for the ASSURE trial was men with baseline HDL<40 and women with baseline HDL<45, both of which are considered low according to clinical guidelines. The median baseline HDL for all patients was 39 mg/dL.

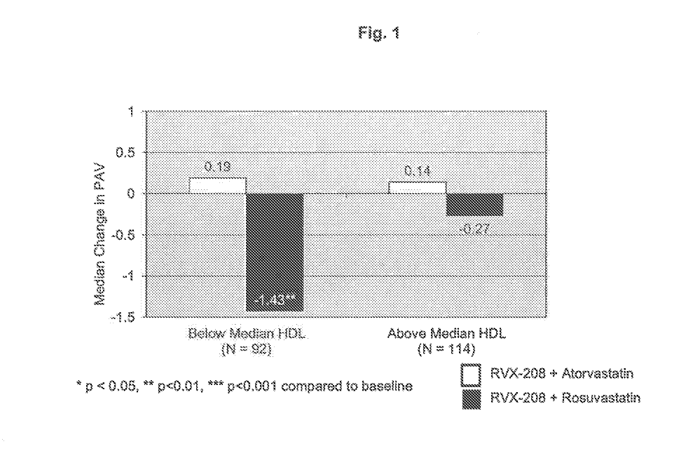

The median change in percent atheroma volume (PAV) patients treated with RVX-208+rosuvastatin as compared to RVX-208+atorvastatin in patients with below median baseline HDL (<39 mg/dL) and patients with above median HDL (>39 mg/dL) was measured. FIG. 1 shows that in patients who began the study with below median HDL, the PAV increased by 0.19% in patients treated with RVX-208+atorvastatin, versus a decrease in PAV by 1.43% in patients treated with RVX-208+rosuvastatin. In patients who began the study with above median HDL, the PAV increased by 0.14% in patients treated with RVX-208+atorvastatin, versus a decrease in PAV by 0.27% in patients treated with RVX-208+rosuvastatin. Treatment with RVX-208+rosuvastatin showed superior effects over treatment with RVX-208+atorvastatin for coronary atherosclerosis (FIG. 1), independent of baseline HDL level.

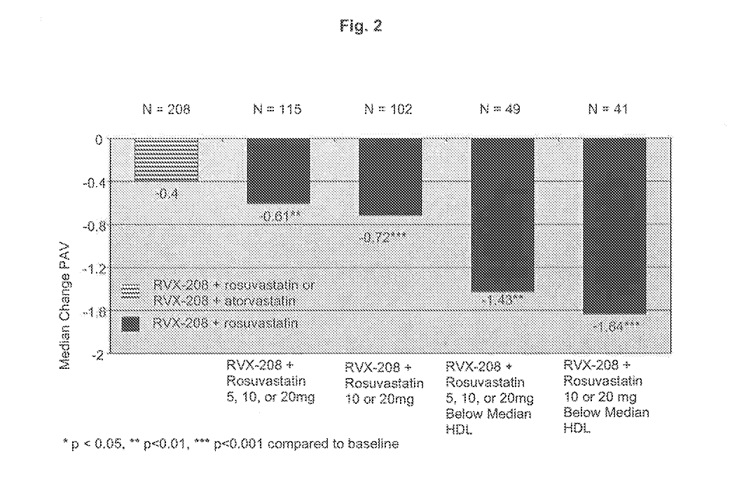

The median change in percent atheroma volume (PAV) was measured in (1) all patients receiving either RVX-208+rosuvastatin or RVX-208+atorvastatin (regardless of baseline HDL values); (2) only those patients receiving RVX-208+rosuvastatin (regardless of baseline HDL values) and (3) only those patients who began the study with below median HDL who were dosed with RVX-208+rosuvastatin (FIG. 2). The most dramatic effects were observed in the group of patients who began the study with below median HDL and who were treated with RVX-208+rosuvastatin (1.64% decrease in PAV).

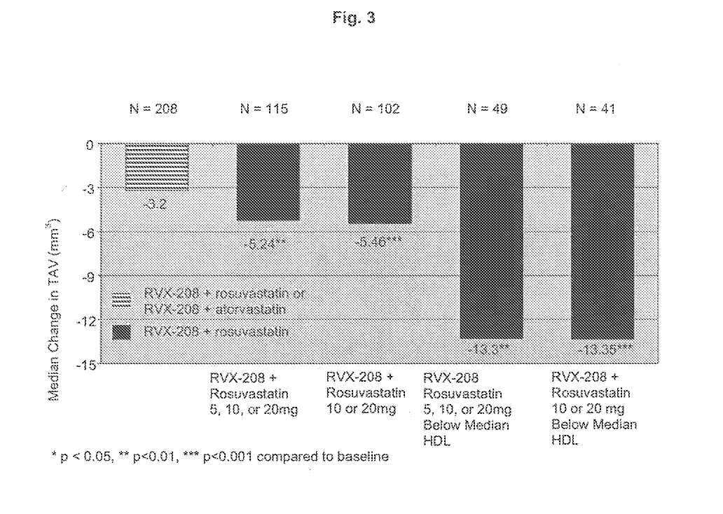

The median change in total atheroma volume (TAV) was measured in (1) all patients dosed with either RVX-208+rosuvastatin or RVX-208+atorvastatin (regardless of baseline HDL values); (2) only those patients dosed with RVX-208+rosuvastatin (regardless of median HDL values), and (3) only those patients who began the study with below median HDL and were dosed with RVX-208+rosuvastatin. (FIG. 3). The most dramatic effects were observed in the group of patients with below median HDL dosed with RVX-208+rosuvastatin (13.35% decrease in TAV).

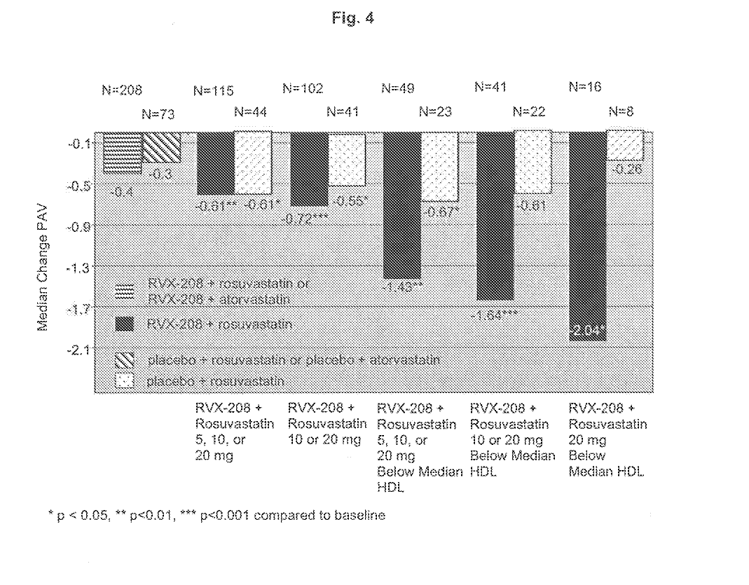

The median change in percent atheroma volume (PAV) was measured in (1) all patients dosed with either RVX-208+rosuvastatin or RVX-208+atorvastatin; (2) patients dosed with placebo 4 rosuvastatin or placebo+atorvastatin; (3) patients dosed with RVX-208+rosuvastatin (regardless of baseline HDL values); (4) patients dosed with placebo+rosuvastatin (regardless of baseline HDL values); (5) patients who began the study with below median HDL and received RVX-208+rosuvastatin; and (6) patients who began the study with below median HDL and received placebo+rosuvastatin (FIG. 4). The most dramatic effects were observed in the group of patients with below median baseline HDL dosed with RVX-208+rosuvastatin (2.04% decrease in PAV).

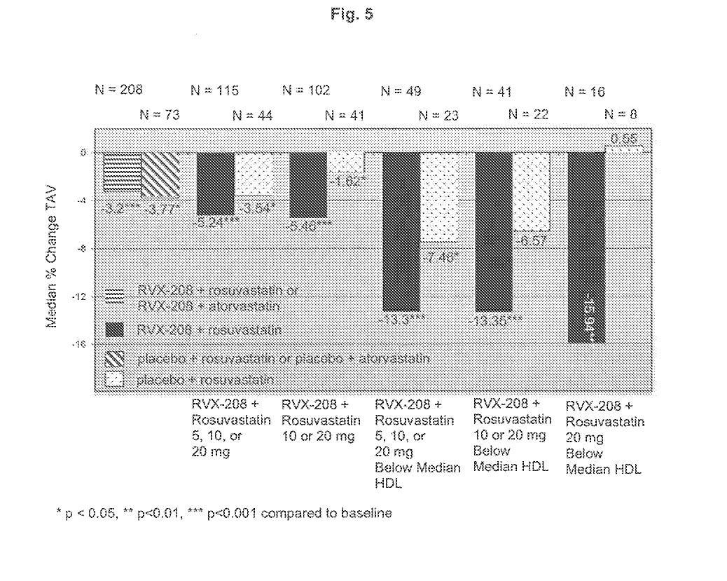

The median change in total atheroma volume (TAV) was measured in (1) all patients dosed with either RVX-208+rosuvastatin or RVX-208+atorvastatin; (2) patients dosed with placebo+rosuvastatin or placebo+atorvastatin; (3) patients dosed with RVX-208+rosuvastatin (regardless of baseline HDL values); (4) patients dosed with placebo+rosuvastatin (regardless of baseline HDL values); (5) patients who began the study with below median HDL and received RVX-208+rosuvastatin; and (6) patients who began the study with below median HDL and received placebo+rosuvastatin (FIG. 5). The most dramatic effects were observed in the group of patients with below median baseline HDL dosed with RVX-208+rosuvastatin (15.95% decrease in TAV). RVX-208+rosuvastatin showed improved effects on coronary atherosclerosis (FIGS. 4 and 5), which was not observed for rosuvastatin alone.

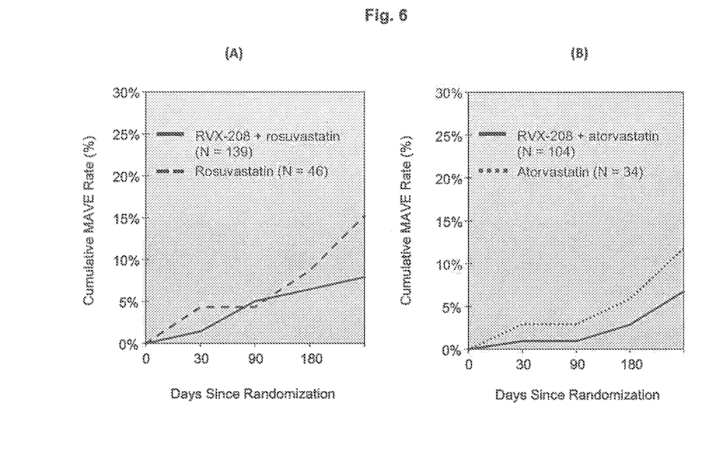

The percentage of major adverse vascular event (MAVE) was measured in patients receiving (1) RVX-208+rosuvastatin as compared to rosuvastatin alone, and (2) for RVX-208+atorvastatin as compared to atorvastatin alone. FIG. 6 shows that the rate of MAVE was lower in patients dosed with RVX-203+rosuvastatin than with rosuvastatin alone, and also that the rate of MAVE was lower in patients dosed with RVX-208+atorvastatin than with atorvastatin alone.

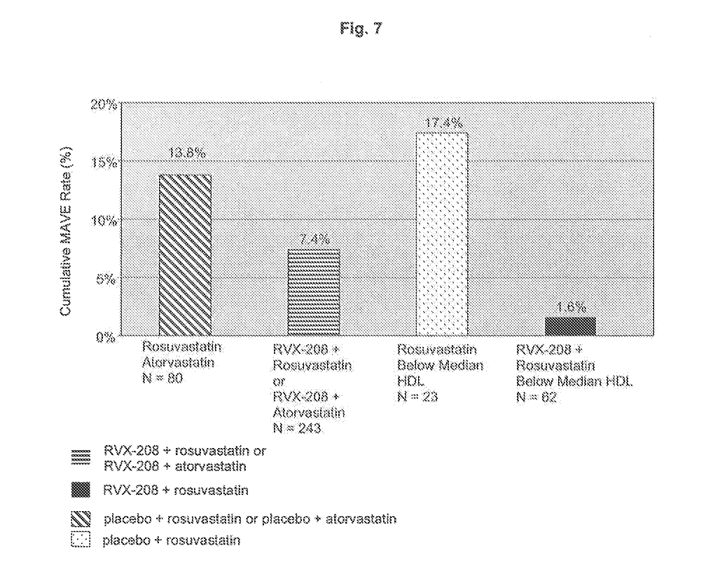

The percentage of MAVEs was measured in (1) patients dosed with either rosuvastatin alone or atorvastatin alone; (2) patients receiving either RVX-208+rosuvastatin or RVX-208+atorvastatin (regardless of baseline HDL values); (3) patients who began the study with below median HDL and were dosed with rosuvastatin+placebo; and (4) patients who began the study with below median HDL and were dosed with RVX-208+rosuvastatin. FIG. 7 shows that the frequency of MAVE in patients treated with either rosuvastatin alone or atorvastatin alone was 13.8%, while the rate of MAVE in patients treated with either RVX-208+rosuvastatin or RVX-208+atorvastatin was 7.4%. In patients with a baseline HDL below 39 mg/dL, the frequency of MAVE was 17.4% in patients treated with either rosuvastatin and atorvastatin alone, while the rate of MAVE was 1.6% in patients treated with either RVX-208+rosuvastatin or RVX-208+atorvastatin. RVX-208+rosuvastatin treatment also reduced adverse vascular events more than RVX-208+atorvastatin (FIG. 6), and this effect was even more pronounced in the below median baseline HDL-c population (FIG. 7).

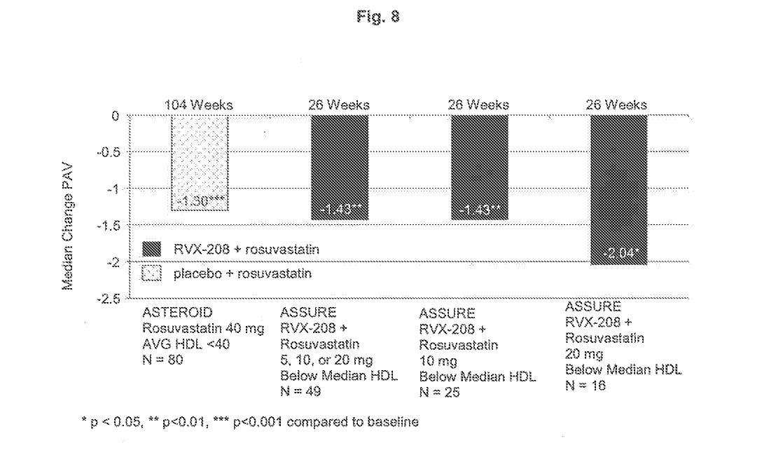

The median change in percent atheroma volume (PAV) was measured (1) in patients dosed with either rosuvastatin alone and (2) in patients receiving RVX-208+various dosages of rosuvastatin. FIG. 8 shows that patients dosed with 40 mg of rosuvastatin without RVX-208 had a PAV decrease of 1.30% after 104 weeks. Patients dosed with RVX-208+all dosages of rosuvastatin (5+10+20 mg), experienced a decrease in PAV of 1.43% after 26 weeks. In patients dosed with RVX-208+rosuvastatin (10 mg), the PAV decreased by 1.43% after 26 weeks. In patients dosed with RVX-208+rosuvastatin (20 mg), the PAV decreased by 2.04% after 26 weeks.

These data show that RVX-208+rosuvastatin treatment was most effective on coronary atherosclerosis in the below median baseline HDL-c population (<39 mg/dL), where rosuvastatin alone was not as effective. Compared to an earlier IVUS-trial (ASTEROID), the RVX-208+rosuvastatin combination in the below median baseline HDL-c population was more effective in reducing coronary atherosclerosis in a shorter time (6 months) and at a lower dose (20 mg) of rosuvastatin (FIG. 8).

All documents, publication, manuals, article, patents, summaries, references and other materials cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed is:

1. A pharmaceutical composition comprising rosuvastatin or pitavastatin or a pharmaceutically acceptable salt of rosuvastatin or pitavastatin and a compound of Formula I 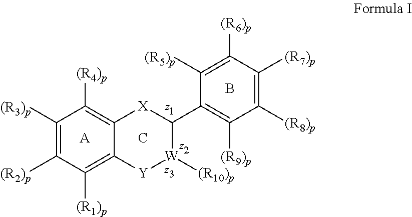
or a pharmaceutically acceptable salt thereof
wherein:
X is N;
Y is CO;
R₁ and R₃ are each independently selected from alkoxy, alkyl, amino, halogen, and hydrogen;
R₂ is selected from alkoxy, alkyl, alkenyl, alkynyl, amide, amino, halogen, and hydrogen;
R₆ and R₈ are each independently selected from alkyl, alkoxy, amino, halogen, and hydrogen;
R₅ and R₉ are each hydrogen;
R₇ is selected from amino, amide, alkyl, hydroxyl, and alkoxy;
R₁₀ is hydrogen;
each W is independently selected from C and N, wherein if W is N, then p is 0 or 1, and if W is C, then p is 1;
for W—(R₁₀)ₚ, W is N and p is 1;
for W—(R₇)ₚ, W is C and p is 1;
for W—(R₄)ₚ, W is C, p is 1 and R₄ is H, or W is N and p is 0;
for (R₁)ₚ, p is 1;
for (R₂)ₚ, p is 1;
for (R₃)ₚ, p is 1;
for (R₆)ₚ, p is 1;
for (R₈)ₚ, p is 1;
for (R₉)ₚ, p is 1;
Z₁ is a double bond, and Z₂ and Z₃ are each a single bond;
with the proviso that if R₁ is hydrogen, then R₃ is alkoxy;
with the proviso that if R₃ is hydrogen, then R₁ is selected from amino and alkoxy;
with the proviso that if R₇ is selected from alkyl, hydroxyl, and alkoxy, then at least one of R₆ and R₈ is independently selected from alkyl, alkoxy, amino, and halogen.

2. The pharmaceutical composition of claim 1, comprising 5-20 mg of rosuvastatin or a pharmaceutically acceptable salt thereof and 100-300 mg of a compound of Formula I or a pharmaceutically acceptable salt thereof.

3. The pharmaceutical composition of claim 1, comprising 5 mg rosuvastatin or a pharmaceutically acceptable salt thereof and 100 mg of a compound of Formula I or a pharmaceutically acceptable salt thereof.

4. The pharmaceutical composition of claim 1, comprising 5 mg rosuvastatin or a pharmaceutically acceptable salt thereof and 200 mg of a compound of Formula I or a pharmaceutically acceptable salt thereof.

5. The pharmaceutical composition of claim 1, comprising 10 mg rosuvastatin or a pharmaceutically acceptable salt thereof and 100 mg of a compound of Formula I or a pharmaceutically acceptable salt thereof.

6. The pharmaceutical composition of claim 1, comprising 10 mg rosuvastatin or a pharmaceutically acceptable salt thereof and 200 mg of a compound of Formula I or a pharmaceutically acceptable salt thereof.

7. The pharmaceutical composition of claim 1, comprising 15 mg rosuvastatin or a pharmaceutically acceptable salt thereof and 100 mg of a compound of Formula I or a pharmaceutically acceptable salt thereof.

8. The pharmaceutical composition of claim 1, comprising 15 mg rosuvastatin or a pharmaceutically acceptable salt thereof and 200 mg of a compound of Formula I or a pharmaceutically acceptable salt thereof.

9. The pharmaceutical composition of claim 1, comprising 20 mg rosuvastatin or a pharmaceutically acceptable salt thereof and 100 mg of a compound of Formula I or a pharmaceutically acceptable salt thereof.

10. The pharmaceutical composition of claim 1, comprising 20 mg rosuvastatin or a pharmaceutically acceptable salt thereof and 200 mg of a compound of Formula I or a pharmaceutically acceptable salt thereof.

11. The pharmaceutical composition of claim 1, comprising 20 mg rosuvastatin or a pharmaceutically acceptable salt thereof and 300 mg of a compound of Formula I or a pharmaceutically acceptable salt thereof.

12. The pharmaceutical composition of any one of claims 1-11, wherein the pharmaceutically acceptable salt of rosuvastatin is rosuvastatin calcium.

13. The pharmaceutical composition of claim 1, comprising 1.0-4.0 mg of pitavastatin or a pharmaceutically acceptable salt thereof and 100-300 mg of a compound of Formula I or a pharmaceutically acceptable salt thereof.

14. The pharmaceutical composition of claim 1, comprising 1 mg pitavastatin or a pharmaceutically acceptable salt thereof and 100 mg of a compound of Formula I or a pharmaceutically acceptable salt thereof.

15. The pharmaceutical composition of claim 1, comprising 1 mg pitavastatin or a pharmaceutically acceptable salt thereof and 200 mg of a compound of Formula I or a pharmaceutically acceptable salt thereof.

16. The pharmaceutical composition of claim 1, comprising 2 mg pitavastatin or a pharmaceutically acceptable salt thereof and 100 mg of a compound of Formula I or a pharmaceutically acceptable salt thereof.

17. The pharmaceutical composition of claim 1, comprising 2 mg pitavastatin or a pharmaceutically acceptable salt thereof and 200 mg of a compound of Formula I or a pharmaceutically acceptable salt thereof.

18. The pharmaceutical composition of claim 1, comprising 4 mg pitavastatin or a pharmaceutically acceptable salt thereof and 100 mg of a compound of Formula I or a pharmaceutically acceptable salt thereof.

19. The pharmaceutical composition of claim 1, comprising 4 mg pitavastatin or a pharmaceutically acceptable salt thereof and 200 mg of a compound of Formula I or a pharmaceutically acceptable salt thereof.

20. The pharmaceutical composition of claim 1, comprising 4 mg pitavastatin or a pharmaceutically acceptable salt thereof and 300 mg of a compound of Formula I or a pharmaceutically acceptable salt thereof.

21. The pharmaceutical composition of any one of claims 13-20, wherein the pharmaceutically acceptable salt of pitavastatin is pitavastatin calcium.

22. A method of treating or preventing a cholesterol- or lipid-related disorder comprising co-administering therapeutically effective amounts of rosuvastatin or pitavastatin or a pharmaceutically acceptable salt of rosuvastatin or pitavastatin and a compound of Formula I 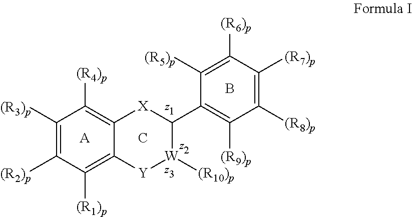
or a pharmaceutically acceptable salt thereof
wherein:
X is N:
Y is CO;
R₁ and R₃ are each independently selected from alkoxy, alkyl, amino, halogen, and hydrogen;
R₂ is selected from alkoxy, alkyl, alkenyl, alkynyl, amide, amino, halogen, and hydrogen;
R₆ and R₈ are each independently selected from alkyl, alkoxy, amino, halogen, and hydrogen;
R₅ and R₉ are each hydrogen;
R₇ is selected from amino, amide, alkyl, hydroxyl, and alkoxy;
R₁₀ is hydrogen;
each W is independently selected from C and N, wherein if W is N, then p is 0 or 1, and if W is C, then p is 1;
for W—(R₁₀)ₚ, W is N and p is 1;
for W—(R₇)ₚ, W is C and p is 1;
for W—(R₄)ₚ, W is C, p is 1 and R₄ is H, or W is N and p is 0;
for (R₁)ₚ, p is 1;
for (R₂)ₚ, p is 1;
for (R₃)ₚ, p is 1;
for (R₆)ₚ, p is 1;
for (R₈)ₚ, p is 1;
for (R₉)ₚ, p is 1;
Z₁ is a double bond, and Z₂ and Z₃ are each a single bond;
with the proviso that if R₁ is hydrogen, then R₃ is alkoxy;
with the proviso that if R₃ is hydrogen, then R₁ is selected from amino and alkoxy;
with the proviso that if R₇ is selected from alkyl, hydroxyl, and alkoxy, then at least one of R₆ and R₈ is independently selected from alkyl, alkoxy, amino, and halogen.

23. The method of claim 22, wherein the disorder is a cardiovascular disorder.

24. The method of claim 22, wherein the disorder is a cerebrovascular disorder.

25. The method of claim 22, wherein the disorder is a peripheral vascular disorder.

26. The method of claim 22, wherein the disorder is a renal bed vascular disorder.

27. The method of claim 22, wherein the cholesterol or lipid related disorder is a metabolic disorder.

28. The method of any one of claims 22-27, wherein disorder is atherosclerosis.

29. The method according to claim 28, wherein co-administration of the therapeutically effective amounts of rosuvastatin or pitavastatin or a pharmaceutically acceptable salt of rosuvastatin or pitavastatin and a compound of Formula I or a pharmaceutically acceptable salt thereof reduces atherosclerosis in the patient.

30. The method according to claim 28 or claim 29, wherein co-administration of the therapeutically effective amounts of rosuvastatin or pitavastatin or a pharmaceutically acceptable salt of rosuvastatin or pitavastatin and a compound of Formula I or a pharmaceutically acceptable salt thereof inhibits or delays progression of atherosclerosis in the patient.

31. The method according to any one of claims 28-30, wherein co-administration of the therapeutically effective amounts of rosuvastatin or pitavastatin or a pharmaceutically acceptable salt thereof and a compound of Formula I or a pharmaceutically acceptable salt thereof reduces percent atheroma volume and/or reduces total atheroma volume.

32. The method according to any one of claims 28-31, wherein co-administration of the therapeutically effective amounts of rosuvastatin or pitavastatin or a pharmaceutically acceptable salt of rosuvastatin or pitavastatin and a compound of Formula I or a pharmaceutically acceptable salt thereof decreases the incidence of major adverse vascular events in the patient.

33. The method of any one of claims 22 to 32, wherein the therapeutically effective amount of rosuvastatin or a pharmaceutically acceptable salt thereof is 5-20 mg and the therapeutically effective amount of a compound of Formula I or a pharmaceutically acceptable salt thereof is 100-300 mg.

34. The method of any one of claims 22 to 33, wherein the therapeutically effective amount of rosuvastatin or a pharmaceutically acceptable salt thereof is 5 mg and the therapeutically effective amount of a compound of Formula I or a pharmaceutically acceptable salt thereof is 100 mg.

35. The method of any one of claims 22 to 33, wherein the therapeutically effective amount of rosuvastatin or a pharmaceutically acceptable salt thereof is 5 mg and the therapeutically effective amount of a compound of Formula I or a pharmaceutically acceptable salt thereof is 200 mg.

36. The method of any one of claims 22 to 33, wherein the therapeutically effective amount of rosuvastatin or a pharmaceutically acceptable salt thereof is 10 mg and the therapeutically effective amount of a compound of Formula I or a pharmaceutically acceptable salt thereof is 100 mg.

37. The method of any one of claims 22 to 33, wherein the therapeutically effective amount of rosuvastatin or a pharmaceutically acceptable salt thereof is 10 mg and the therapeutically effective amount of a compound of Formula I or a pharmaceutically acceptable salt thereof is 200 mg.

38. The method of any one of claims 22 to 33, wherein the therapeutically effective amount of rosuvastatin or a pharmaceutically acceptable salt thereof is 15 mg and the therapeutically effective amount of a compound of Formula I or a pharmaceutically acceptable salt thereof is 200 mg.

39. The method of any one of claims 22 to 33, wherein the therapeutically effective amount of rosuvastatin or a pharmaceutically acceptable salt thereof is 20 mg and the therapeutically effective amount of a compound of Formula I or a pharmaceutically acceptable salt thereof is 200 mg.

40. The method of any one of claims 22 to 33, wherein the therapeutically effective amount of rosuvastatin or a pharmaceutically acceptable salt thereof is 20 mg and the therapeutically effective amount of a compound of Formula I or a pharmaceutically acceptable salt thereof is 300 mg.

41. The method of any one of claims 22 to 33, wherein rosuvastatin or a pharmaceutically acceptable salt thereof and a compound of Formula I or a pharmaceutically acceptable salt thereof are administered as a single composition.

42. The method of any one of claims 22 to 40, wherein rosuvastatin or a pharmaceutically acceptable salt thereof and a compound of Formula I or a pharmaceutically acceptable salt thereof are administered as separate compositions.

43. The method of any one of claims 22 to 42, wherein rosuvastatin or a pharmaceutically acceptable salt thereof and a compound of Formula I or a pharmaceutically acceptable salt thereof are administered once daily.

44. The method of any one of claims 22-42, wherein rosuvastatin or a pharmaceutically acceptable salt thereof and a compound of Formula I or a pharmaceutically acceptable salt thereof are administered twice daily.

45. The method of any one of claims 22-42, wherein rosuvastatin or a pharmaceutically acceptable salt thereof is administered once daily and a compound of Formula I or a pharmaceutically acceptable salt is administered twice daily.

46. The method of any one of claims 22-45, wherein the patient has a HDL of <39 mg/dL at the initiation of treatment with rosuvastatin or a pharmaceutically acceptable salt thereof and a compound of Formula I or a pharmaceutically acceptable salt thereof.

47. The method of claim 46, comprising co-administering 10-20 mg/day rosuvastatin or a pharmaceutically acceptable salt thereof and 200 mg/day of a compound of Formula I or a pharmaceutically acceptable salt thereof.

48. The method of claim 46, comprising co-administering 20 mg/day rosuvastatin or a pharmaceutically acceptable salt thereof and 200 mg/day of a compound of Formula I or a pharmaceutically acceptable salt thereof.

49. The method of any one of claims 22-48, wherein the pharmaceutically acceptable salt of rosuvastatin is rosuvastatin calcium.

50. The method of any one of claims 22 to 32, wherein the therapeutically effective amount of pitavastatin or a pharmaceutically acceptable salt thereof is 1-4 mg and the therapeutically effective amount of a compound of Formula I or a pharmaceutically acceptable salt thereof is 100-300 mg.

51. The method of any one of claims 22 to 32, wherein the therapeutically effective amount of pitavastatin or a pharmaceutically acceptable salt thereof is 1 mg and the therapeutically effective amount of a compound of Formula I or a pharmaceutically acceptable salt thereof is 100 mg.

52. The method of any one of claims 22 to 32, wherein the therapeutically effective amount of pitavastatin or a pharmaceutically acceptable salt thereof is 1 mg and the therapeutically effective amount of a compound of Formula I or a pharmaceutically acceptable salt thereof is 200 mg.

53. The method of any one of claims 22 to 32, wherein the therapeutically effective amount of pitavastatin or a pharmaceutically acceptable salt thereof is 2 mg and the therapeutically effective amount of a compound of Formula I or a pharmaceutically acceptable salt thereof is 100 mg.

54. The method of any one of claims 22 to 32, wherein the therapeutically effective amount of pitavastatin or a pharmaceutically acceptable salt thereof is 2 mg and the therapeutically effective amount of a compound of Formula I or a pharmaceutically acceptable salt thereof is 200 mg.

55. The method of any one of claims 22 to 32, wherein the therapeutically effective amount of pitavastatin or a pharmaceutically acceptable salt thereof is 4 mg and the therapeutically effective amount of a compound of Formula I or a pharmaceutically acceptable salt thereof is 100 mg.

56. The method of any one of claims 22 to 32, wherein the therapeutically effective amount of pitavastatin or a pharmaceutically acceptable salt thereof is 4 mg and the therapeutically effective amount of a compound of Formula I or a pharmaceutically acceptable salt thereof is 200 mg.

57. The method of any one of claims 22 to 32, wherein the therapeutically effective amount of pitavastatin or a pharmaceutically acceptable salt thereof is 4 mg and the therapeutically effective amount of a compound of Formula I or a pharmaceutically acceptable salt thereof is 300 mg.

58. The method of any one of claims 22 to 32 or 50 to 57, wherein pitavastatin or a pharmaceutically acceptable salt thereof and a compound of Formula I or a pharmaceutically acceptable salt thereof are administered as a single composition.

59. The method of any one of claims 22 to 32 or 50 to 57, wherein pitavastatin or a pharmaceutically acceptable salt thereof and a compound of Formula I or a pharmaceutically acceptable salt thereof are administered as separate compositions.

60. The method of any one of claims 22 to 32 or 50 to 59, wherein pitavastatin or a pharmaceutically acceptable salt thereof and a compound of Formula I or a pharmaceutically acceptable salt thereof are administered once daily.

61. The method of any one of claims 22 to 32 or 50 to 59, wherein pitavastatin or a pharmaceutically acceptable salt thereof and a compound of Formula I or a pharmaceutically acceptable salt thereof are administered twice daily.

62. The method of any one of claims 22 to 32 or 50 to 59, wherein pitavastatin or a pharmaceutically acceptable salt thereof is administered once daily and a compound of Formula I or a pharmaceutically acceptable salt is administered twice daily.

63. The method of any one of claims 22 to 32 or 50 to 62, wherein the patient has a HDL of <39 mg/dL at the initiation of treatment with pitavastatin or a pharmaceutically acceptable salt thereof and a compound of Formula I or a pharmaceutically acceptable salt thereof.

64. The method of claim 63, comprising co-administering 1-4 mg/day pitavastatin or a pharmaceutically acceptable salt thereof and 200 mg/day of a compound of Formula I or a pharmaceutically acceptable salt thereof.

65. The method of claim 63, comprising co-administering 4 mg/day pitavastatin or a pharmaceutically acceptable salt thereof and 200 mg/day of a compound of Formula I or a pharmaceutically acceptable salt thereof.

66. The method of any one of claims 22-32 or 50-65, wherein the pharmaceutically acceptable salt of pitavastatin is pitavastatin calcium.

67. The pharmaceutical composition of any one of claims 1-21 or the method of any one of claims 22-66, wherein the compound of Formula I is selected from:
2-(3,5-dimethyl-4-(2-morpholinoethoxy)phenyl)-5,7-dimethoxyquinazolin-4(3H)-one;
2-(3,5-di-tert-butyl-4-hydroxyphenyl)-5,7-dimethoxyquinazolin-4(3H)-one;
2-(4-hydroxy-3-methoxyphenyl)-5,7-dimethoxyquinazolin-4(3H)-one;
2-(3-chloro-4-hydroxyphenyl)-5,7-dimethoxyquinazolin-4(3H)-one;
5,7-dimethoxy-2-(4-(4-methylpiperazin-1-yl)phenyl)quinazolin-4(3H)-one;
2-(4-hydroxy-3,5-dimethylphenyl)-6,7-dimethoxyquinazolin-4(3H)-one;
2-(4-(6,7-dimethoxy-4-oxo-3, 4-dihydroquinazolin-2-yl)-2,6-dimethylphenoxy)acetamide;
2-(3-chloro-4-(2-hydroxyethoxy)phenyl)-5,7-dimethoxyquinazolin-4(3H)-one;
2-(4-(2-hydroxyethoxy)-3-methoxyphenyl)-5,7-dimethoxyquinazolin-4(3H)-one;
2-(4-(2-hydroxyethoxy)-3,5-dimethylphenyl)-6,7-dimethoxyquinazolin-4(3H)-one;
N-(4-(5,7-dimethoxy-4-oxo-3,4-dihydroquinazolin-2-yl)-2,6-dimethylphenyl)-2-hydroxyacetamide;
2-(4-(5,7-dimethoxy-4-oxo-3,4-dihydroquinazolin-2-yl)-2,6-dimethylphenoxy)acetic acid;
N-(4-(5,7-dimethoxy-4-oxo-3,4-dihydroquinazolin-yl)phenyl)-2-hydroxyacetamide;
2-(4-hydroxy-3,5-dimethylphenyl)-5,7-dimethoxypyrido[2,3-d]pyrimidin-4(3H)-one;
5,7-dimethoxy-2-(4-methoxy-3-(morpholinomethyl)phenyl)quinazolin-4(3H)-one;
2-(3,5-dimethyl-4-(2-(pyrrolidin-1-yl)ethoxy)phenyl)-5,7-dimethoxyquinazolin-4(3H)-one;
2-(4-hydroxy-3-(2-hydroxyethyl)phenyl)-5,7-dimethoxyquinazolin-4(3H)-one;
2-(4-(bis(2-hydroxyethyl)amino)phenyl)-5,7-dimethoxy-pyrido[2,3-d]pyrimidin-4(3H)-one;
5,7-dimethoxy-2-(4-(2-methoxyethoxy)-3,5-dimethylphenyl)quinazolin-4(3H)-one;
2-(4-(2-hydroxyethoxy)-3,5-dimethylphenyl)-5-methoxyquinazolin-4(3H)-one;
(E)-N′-(4-(5,7-dimethoxy-4-oxo-3,4-dihydroquinazolin-2-yl)-2,6-dimethylphenyl)-N,N-dimethylformimidamide;
2-(4-benzyloxy)-3,5-dimethylphenyl)-5,7-dimethoxyquinazolin-4(3H)-one;
2-(4-(2-aminoethoxy)-3,5-dimethylphenyl)-5,7-dimethoxyquinazolin-4(3H)-one;
2-(4-(5,7-dimethoxy-4-oxo-3,4-dihydroquinazolin-2-yl)-2-methoxyphenoxy)acetic acid;
2-(4-(5,7-dimethoxy-4-oxo-3,4-dihydroquinazolin-2-yl)-2,6-dimethyl-phenoxy)ethyl propylcarbamate;
2-(4-(5,7-dimethoxy-4-oxo-3,4-dihydroquinazolin-2-yl)-2,6-dimethyl-phenoxy)ethyl methylcarbamate;
N-(2-(4-(5,7-dimethoxy-4-oxo-3,4-dihydroquinazolin-2-yl)-2,6-dimethylphenoxy)ethyl)-4-methylbenzamide;
N-(2-(4-(5,7-dimethoxy-4-oxo-3,4-dihydroquinazolin-2-yl)-2,6-dimethylphenoxy)ethyl)benzenesulfonamide;
N-(2-(4-(5,7-dimethoxy-4-oxo-3,4-dihydroquinazolin-2-yl)-2,6-dimethylphenoxy)ethyl-4-methylbenzenesulfonamide;
N-(2-(4-(5,7-dimethoxy-4-oxo-3,4-dihydroquinazolin-2-yl)-2,6-dimethylphenoxy)ethyl)-4-methoxybenzamide;
N-(2-(4-(5,7-dimethoxy-4-oxo-3,4-dihydroquinazolin-2-yl)-2,6-dimethylphenoxy)ethyl)benzamide;
1-(2-(4-(5,7-dimethoxy-4-oxo-3,4-dihydroquinazolin-2-yl)-2,6-dimethylphenoxy)ethyl)-3-methylurea;
1-(2-(4-(5,7-dimethoxy-4-oxo-3,4-dihydroquinazolin-2-yl)-2,6-dimethylphenoxy)ethyl)-3-(4-methoxyphenyl)urea;
2-(3,5-dimethyl-4-(2-morpholinoethoxy)phenyl)quinazolin-4(3H)-one;
2-(4-(2-hydroxyethoxy)-3,5-dimethylphenyl)quinazolin-4(3H)-one;
2-(2,3-dihydrobenzo[b][1,4]dioxin-6-yl)quinazolin-4(3H)-one;
2-(4-(4-oxo-3,4-dihydroquinazolin-2-yl)phenoxy)acetic acid;
2-(4-(dimethylamino)naphthalen-1-yl)quinazolin-4(3H)-one;
2-(4-(4-oxo-3,4-dihydroquinazolin-2-yl)phenoxy)acetamide;
2-(4-(bis(2-hydroxyethyl)amino)phenyl)quinazolin-4(3H)-one;
2-(4-(5,7-dimethoxyquinazolin-2-yl)-2,6-dimethylphenoxy)ethanol;
2-(4-(2-hydroxyethoxy)-3,5-dimethylphenyl)-5,7-dimethylquinazolin-4(3H)-one;
5,7-dichloro-2-(4-(2-hydroxyethoxy)-3,5-dimethylphenyl)quinazolin-4(3H)-one;
6-bromo-2-(4-hydroxy-3,5-dimethylphenyl)quinazolin-4(3H)-one;
6-bromo-2-(4-(2-hydroxyethoxy)-3,5-dimethylphenyl)quinazolin-4(3H)-one;
6-bromo-2-(4-(2-(tert-butyldimethylsilyloxy)ethoxy)-3,5-dimethylphenyl)quinazolin-4(3H)-one;
5,7-dimethoxy-2-(pyridin-4-yl)quinazolin-4(3H)-one;
2-(4-(dimethylamino)naphthalen-1-yl)-5,7-dimethoxyquinazolin-4(3H)-one;
2-(4-(dimethylamino)pyridin-1-yl)-6,7-dimethoxyquinazolin-4(3H)-one;
2-(4-hydroxy-3,5-dimethylphenyl)-5,7-dimethoxy-1-methylquinazolin-4(3H)-one;
2-(4-hydroxy-3,5-dimethylphenyl)-5,7-dimethoxy-6-(morphoinomethyl)quinazolin-4(3H)-one;
2-(4-(2-hydroxyethoxy)-3,5-dimethylphenyl)-6-methoxyquinazolin-4(3H)-one;
5-hydroxy-2-(4-hydroxy-3,5-dimethylphenyl)-7-methoxyquinazolin-4(3H)-one;
3-(4-hydroxy-3,5-dimethylphenyl)-6,8-dimethoxyisoquinolin-1(2H)-one;
3-(4-(2-hydroxyethoxy)-3,5-dimethylphenyl)-6,8-dimethoxyisoquinolin-1(2H)-one;
2-(4-hydroxy-3,5-dimethylphenyl)-5,7-dimethoxyquinazolin-4(3H)-one;
2-(4-(2-hydroxyethoxy)-3,5-dimethylphenyl)-5,7-dimethoxyquinazolin-4(3H) one);
2-(4-hydroxy-3-methoxyphenyl)-5,7-dimethoxyquinazolin-4(3H)-one;
2-(2,3-dihydrobenzo[b][1,4]dioxin-6-yl)-5,7-dimethoxyquinazolin-4(3H)-one;
2-(4-(bis(2-hydroxyethyl)amino)phenyl)-5,7-dimethoxyquinazolin-4(3H)-one;
2-(4-(bis(2-hydroxyethyl)amino)phenyl)-6,7-dimethoxyquinazolin-4(3H)-one;
2-(2,3-dihydrobenzo[b][1,4]dioxin-6-yl)-6,7-dimethoxyquinazolin-4(3H)-one;
2-(4-((4-ethylpiperazin-1-yl)methyl)phenyl)-5,7-dimethoxyquinazolin-4(3H)-one;
2-(4-(2-hydroxyethoxy)-3,5-dimethylphenyl)-5,7-dimethoxypyrido[2,3-d]pyrimidin-4(3H)-one;
2-(2-chloro-6-methylpyridin-4-yl)-5,7-dimethoxyquinazolin-4(3H)-one;
5,7-dimethoxy-2-(4-methoxy-3,5-dimethylphenyl)quinazolin-4(3H)-one;
2-(4-amino-3,5-dimethylphenyl)-5,7-dimethoxyquinazolin-4(3H)-one;
N1-(2-(4-(5,7-dimethoxy-4-oxo-3,4-dihydroquinazolin-2-yl)-2,6-dimethylphenoxy)ethyl)-N2-methylphthalamide;
4-chloro-N-(2-(4-(5,7-dimethoxy-4-oxo-3,4-dihydroquinazolin-2-yl)-2,6-dimethylphenoxy)ethyl)benzenesulfonamide;
3-(4-(5,7-dimethoxy-4-oxo-3,4-dihydroquinazolin-2-yl)phenyl)propanoic acid;
5,7-dimethoxy-2-(4-((4-methylpiperazin-1-yl)methyl)phenyl)quinazolin-4(3H)-one;
5,7-dimethoxy-2-(4-(morpholinomethyl)phenyl)quinazolin-4(3H)-one;
5,7-dimethoxy-2-(4-morpholinophenyl)quinazolin-4(3H)-one;
2-(4-(5,7-dimethoxy-4-oxo-3,4-dihydroquinazolin-2-yl)-2,6-dimethylphenoxy)ethyl cyclohexylcarbamate;
N-(2-(4-(5,7-dimethoxy-4-oxo-3,4-dihydroquinazolin-2-yl)-2,6-dimethylphenoxy)ethyl)acetamide;
N-(2-(4-(5,7-dimethoxy-4-oxo-3,4-dihydroquinazolin-2-yl)-2,6-dimethylphenoxy)ethyl)isobutyramide;
1-(2-(4-(5,7-dimethoxy-4-oxo-3,4-dihydroquinazolin-2-yl)-2,6-dimethylphenoxy)ethyl)-3-phenylurea; and
3-(2-(4-(5,7-dimethoxy-4-oxo-3,4-dihydroquinazolin-2-yl)-2,6-dimethylphenoxy)ethyl)-1,1-dimethylurea.

68. Use of rosuvastatin or pitavastatin or a pharmaceutically acceptable salt of rosuvastatin or pitavastatin and a compound of Formula I 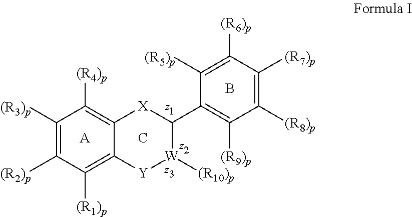
or a pharmaceutically acceptable salt thereof in the manufacture of a medicament for treating or preventing atherosclerosis or other cholesterol- or lipid-related disorder,
wherein:
X is N;
Y is CO;
R₁ and R₃ are each independently selected from alkoxy, alkyl, amino, halogen, and hydrogen;
R₂ is selected from alkoxy, alkyl, alkenyl, alkynyl, amide, amino, halogen, and hydrogen;
R₆ and R₈ are each independently selected from alkyl, alkoxy, amino, halogen, and hydrogen;
R₅ and R₉ are each hydrogen;
R₇ is selected from amino, amide, alkyl, hydroxyl, and alkoxy;
R₁₀ is hydrogen;
each W is independently selected from C and N, wherein if W is N, then p is 0 or 1, and if W is C, then p is 1;
for W—(R₁₀)ₚ, W is N and p is 1;
for W—(R₇)ₚ, W is C and p is 1;
for W—(R₄)ₚ, W is C, p is 1 and R₄ is H, or W is N and p is 0;
for (R₁)ₚ, p is 1;
for (R₂)ₚ, p is 1;
for (R₃)ₚ, p is 1;
for (R₆)ₚ, p is 1;
for (R₈)ₚ, p is 1;
for (R₉)ₚ, p is 1;
Z₁ is a double bond, and Z₂ and Z₃ are each a single bond;
with the proviso that if R₁ is hydrogen, then R₃ is alkoxy;
with the proviso that if R₃ is hydrogen, then R₁ is selected from amino and alkoxy;
with the proviso that if R₇ is selected from alkyl, hydroxyl, and alkoxy, then at least one of R₆ and R₈ is independently selected from alkyl, alkoxy, amino, and halogen.

69. Use of rosuvastatin or pitavastatin or a pharmaceutically acceptable salt of rosuvastatin or pitavastatin and a compound of Formula I 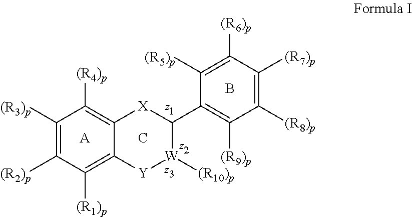
or a pharmaceutically acceptable salt thereof for treating or preventing a cholesterol- or lipid-related disorder,
wherein:
X is N;
Y is CO;
R₁ and R₃ are each independently selected from alkoxy, alkyl, amino, halogen, and hydrogen;
R₂ is selected from alkoxy, alkyl, alkenyl, alkynyl, amide, amino, halogen, and hydrogen;
R₆ and R₈ are each independently selected from alkyl, alkoxy, amino, halogen, and hydrogen;
R₅ and R₉ are each hydrogen;
R₇ is selected from amino, amide, alkyl, hydroxyl, and alkoxy;
R₁₀ is hydrogen;
each W is independently selected from C and N, wherein if W is N, then p is 0 or 1, and if W is C, then p is 1;
for W—(R₁₀)ₚ, W is N and p is 1;
for W—(R₇)ₚ, W is C and p is 1;
for W—(R₄)ₚ, W is C, p is 1 and R₄ is H, or W is N and p is 0;
for (R₁)ₚ, p is 1;
for (R₂)ₚ, p is 1;
for (R₃)ₚ, p is 1;
for (R₆)ₚ, p is 1;
for (R₈)ₚ, p is 1;
for (R₉)ₚ, p is 1;
Z₁ is a double bond, and Z₂ and Z₃ are each a single bond;
with the proviso that if R₁ is hydrogen, then R₃ is alkoxy;
with the proviso that if R₃ is hydrogen, then R₁ is selected from amino and alkoxy;
with the proviso that if R₇ is selected from alkyl, hydroxyl, and alkoxy, then at least one of R₆ and R₈ is independently selected from alkyl, alkoxy, amino, and halogen.

70. The use of claim 69, wherein the disorder is a cardiovascular disorder.

71. The use of claim 69, wherein the disorder is a cerebrovascular disorder.

72. The use of claim 69, wherein the disorder is a peripheral vascular disorder.

73. The use of claim 69, wherein the disorder is a renal bed vascular disorder.

74. The use of claim 69, wherein the cholesterol or lipid related disorder is a metabolic disorder.

75. The use of any one of claims 69-74, wherein disorder is atherosclerosis.

76. The use according to claim 75, wherein co-administration of the therapeutically effective amounts of rosuvastatin or pitavastatin or a pharmaceutically acceptable salt of rosuvastatin or pitavastatin and a compound of Formula I or a pharmaceutically acceptable salt thereof reduces atherosclerosis in the patient.

77. The use according to claim 75 or claim 76, wherein co-administration of the therapeutically effective amounts of rosuvastatin or pitavastatin or a pharmaceutically acceptable salt of rosuvastatin or pitavastatin and a compound of Formula I or a pharmaceutically acceptable salt thereof inhibits or delays progression of atherosclerosis in the patient.

78. The use according to any one of claims 75-77, wherein co-administration of the therapeutically effective amounts of rosuvastatin or pitavastatin or a pharmaceutically acceptable salt of rosuvastatin or pitavastatin and a compound of Formula I or a pharmaceutically acceptable salt thereof reduces percent atheroma volume and/or reduces total atheroma volume.

79. The use according to any one of claims 75-77, wherein co-administration of the therapeutically effective amounts of rosuvastatin or pitavastatin or a pharmaceutically acceptable salt of rosuvastatin or pitavastatin and a compound of Formula I or a pharmaceutically acceptable salt thereof decreases the incidence of major adverse vascular events in the patient.

80. The use of any one of claims 69 to 79, wherein the therapeutically effective amount of rosuvastatin or a pharmaceutically acceptable salt thereof is 5-20 mg and the therapeutically effective amount of a compound of Formula I or a pharmaceutically acceptable salt thereof is 100-300 mg.

81. The use of any one of claims 69 to 80, wherein the therapeutically effective amount of rosuvastatin or a pharmaceutically acceptable salt thereof is 5 mg and the therapeutically effective amount of a compound of Formula I or a pharmaceutically acceptable salt thereof is 100 mg.

82. The use of any one of claims 69 to 80, wherein the therapeutically effective amount of rosuvastatin or a pharmaceutically acceptable salt thereof is 5 mg and the therapeutically effective amount of a compound of Formula I or a pharmaceutically acceptable salt thereof is 200 mg.

83. The use of any one of claims 69 to 80, wherein the therapeutically effective amount of rosuvastatin or a pharmaceutically acceptable salt thereof is 10 mg and the therapeutically effective amount of a compound of Formula I or a pharmaceutically acceptable salt thereof is 100 mg.

84. The use of any one of claims 69 to 80, wherein the therapeutically effective amount of rosuvastatin or a pharmaceutically acceptable salt thereof is 10 mg and the therapeutically effective amount of a compound of Formula I or a pharmaceutically acceptable salt thereof is 200 mg.

85. The use of any one of claims 69 to 80, wherein the therapeutically effective amount of rosuvastatin or a pharmaceutically acceptable salt thereof is 15 mg and the therapeutically effective amount of a compound of Formula I or a pharmaceutically acceptable salt thereof is 200 mg.

86. The use of any one of claims 69 to 80, wherein the therapeutically effective amount of rosuvastatin or a pharmaceutically acceptable salt thereof is 20 mg and the therapeutically effective amount of a compound of Formula I or a pharmaceutically acceptable salt thereof is 200 mg.

87. The use of any one of claims 69 to 80, wherein the therapeutically effective amount of rosuvastatin or a pharmaceutically acceptable salt thereof is 20 mg and the therapeutically effective amount of a compound of Formula I or a pharmaceutically acceptable salt thereof is 300 mg.

88. The use of any one of claims 69 to 87, wherein rosuvastatin or a pharmaceutically acceptable salt thereof and a compound of Formula I or a pharmaceutically acceptable salt thereof are administered as a single composition.

89. The use of any one of claims 69 to 87, wherein rosuvastatin or a pharmaceutically acceptable salt thereof and a compound of Formula I or a pharmaceutically acceptable salt thereof are administered as separate compositions.

90. The use of any one of claims 69-88, wherein rosuvastatin or a pharmaceutically acceptable salt thereof and a compound of Formula I or a pharmaceutically acceptable salt thereof are administered once daily.

91. The use of any one of claims 69 to 89, wherein rosuvastatin or a pharmaceutically acceptable salt thereof and a compound of Formula I or a pharmaceutically acceptable salt thereof are administered twice daily.

92. The use of any one of claims 69-89, wherein rosuvastatin or a pharmaceutically acceptable salt thereof is administered once daily and a compound of Formula I or a pharmaceutically acceptable salt is administered twice daily.

93. The use of any one of claims 69-92, wherein the patient has a HDL of <39 mg/dL at the initiation of treatment with rosuvastatin or a pharmaceutically acceptable salt thereof and a compound of Formula I or a pharmaceutically acceptable salt thereof.

94. The use of claim 93, comprising co-administering 10-20 mg/day rosuvastatin or a pharmaceutically acceptable salt thereof and 200 mg/day of a compound of Formula I or a pharmaceutically acceptable salt thereof.

95. The use of claim 93, comprising co-administering 20 mg/day rosuvastatin or a pharmaceutically acceptable salt thereof and 200 mg/day of a compound of Formula I or a pharmaceutically acceptable salt thereof.

96. The use of any one of claims 69-95, wherein the pharmaceutically acceptable salt of rosuvastatin is rosuvastatin calcium.

97. The use of any one of claims 69 to 79, wherein the therapeutically effective amount of pitavastatin or a pharmaceutically acceptable salt thereof is 1-4 mg and the therapeutically effective amount of a compound of Formula I or a pharmaceutically acceptable salt thereof is 100.300 mg.

98. The use of any one of claims 69 to 79, wherein the therapeutically effective amount of pitavastatin or a pharmaceutically acceptable salt thereof is 1 mg and the therapeutically effective amount of a compound of Formula I or a pharmaceutically acceptable salt thereof is 100 mg.

99. The use of any one of claims 69 to 79, wherein the therapeutically effective amount of pitavastatin or a pharmaceutically acceptable salt thereof is 1 mg and the therapeutically effective amount of a compound of Formula I or a pharmaceutically acceptable salt thereof is 200 mg.

100. The use of any one of claims 69 to 79, wherein the therapeutically effective amount of pitavastatin or a pharmaceutically acceptable salt thereof is 2 mg and the therapeutically effective amount of a compound of Formula I or a pharmaceutically acceptable salt thereof is 100 mg.

101. The use of any one of claims 69 to 79, wherein the therapeutically effective amount of pitavastatin or a pharmaceutically acceptable salt thereof is 2 mg and the therapeutically effective amount of a compound of Formula I or a pharmaceutically acceptable salt thereof is 200 mg.

102. The use of any one of claims 69 to 79, wherein the therapeutically effective amount of pitavastatin or a pharmaceutically acceptable salt thereof is 4 mg and the therapeutically effective amount of a compound of Formula I or a pharmaceutically acceptable salt thereof is 100 mg.

103. The use of any one of claims 69 to 79, wherein the therapeutically effective amount of pitavastatin or a pharmaceutically acceptable salt thereof is 4 mg and the therapeutically effective amount of a compound of Formula I or a pharmaceutically acceptable salt thereof is 200 mg.

104. The use of any one of claims 69 to 79, wherein the therapeutically effective amount of pitavastatin or a pharmaceutically acceptable salt thereof is 4 mg and the therapeutically effective amount of a compound of Formula I or a pharmaceutically acceptable salt thereof is 300 mg.

105. The use of any one of claims 69 to 79 or 97-104, wherein pitavastatin or a pharmaceutically acceptable salt thereof and a compound of Formula I or a pharmaceutically acceptable salt thereof are administered as a single composition.

106. The use of any one of claims 69 to 79 or 97-104, wherein pitavastatin or a pharmaceutically acceptable salt thereof and a compound of Formula I or a pharmaceutically acceptable salt thereof are administered as separate compositions.

107. The use of any one of claims 69 to 79 or 97-106, wherein pitavastatin or a pharmaceutically acceptable salt thereof and a compound of Formula I or a pharmaceutically acceptable salt thereof are administered once daily.

108. The use of any one of claims 69 to 79 or 97-106, wherein pitavastatin or a pharmaceutically acceptable salt thereof and a compound of Formula I or a pharmaceutically acceptable salt thereof are administered twice daily.

109. The use of any one of claims 69 to 79 or 97-106, wherein pitavastatin or a pharmaceutically acceptable salt thereof is administered once daily and a compound of Formula I or a pharmaceutically acceptable salt is administered twice daily.

110. The use of any one of claims 69 to 79 or 97-109, wherein the patient has a HDL of <39 mg/dL at the initiation of treatment with pitavastatin or a pharmaceutically acceptable salt thereof and a compound of Formula I or a pharmaceutically acceptable salt thereof.

111. The use of claim 110, comprising co-administering 1-4 mg/day pitavastatin or a pharmaceutically acceptable salt thereof and 200 mg/day of a compound of Formula I or a pharmaceutically acceptable salt thereof.

112. The use of claim 110, comprising co-administering 4 mg/day rosuvastatin or a pharmaceutically acceptable salt thereof and 200 mg/day of a compound of Formula I or a pharmaceutically acceptable salt thereof.

113. The use of any one of claims 69-79 and 97-112, wherein the pharmaceutically acceptable salt of pitavastatin is pitavastatin calcium.

114. The use of any one of claims 69-113, wherein the compound of Formula I is selected from:
2-(3,5-dimethyl-4-(2-morpholinoethoxy)phenyl)-5,7-dimethoxyquinazolin-4(3H)-one;
2-(3,5-di-tert-butyl-4-hydroxyphenyl)-5,7-dimethoxyquinazolin-4(3H)-one;
2-(4-hydroxy-3-methoxyphenyl)-5,7-dimethoxyquinazolin-4(3H)-one;
2-(3-chloro-4-hydroxyphenyl)-5,7-dimethoxyquinazolin-4(3H)-one;
5,7-dimethoxy-2-(4-(4-methylpiperazin-1-yl)phenyl)quinazolin-4(3H)-one;
2-(4-hydroxy-3,5-dimethylphenyl)-7-dimethoxyquinazolin-4(3H)-one;
2-(4-(6,7-dimethoxy-4-oxo-3,4-dihydroquinazolin-2-yl)-2,6-dimethylphenoxy)acetamide;
2-(3-chloro-4-(2-hydroxyethoxy)phenyl)-5,7-dimethoxyquinazolin-4(3H)-one;
2-(4-(2-hydroxyethoxy)-3-methoxyphenyl)-5,7-dimethoxyquinazolin-4(3H)-one;
2-(4-(2-hydroxyethoxy)-3,5-dimethylphenyl)-6,7-dimethoxyquinazolin-4(3H)-one;
N-(4-(5,7-dimethoxy-4-oxo-3,4-dihydroquinazolin-2-yl)-2,6-dimethylphenyl)-2-hydroxyacetamide;
2-(4-(5, 7-dimethoxy-4-oxo-3,4-dihydroquinazolin-2-yl)-2,6-dimethylphenoxy)acetic acid;
N-(4-(5,7-dimethoxy-4-oxo-3,4-dihydroquinazolin-2-yl)phenyl)-2-hydroxyacetamide;
2-(4-hydroxy-3,5-dimethylphenyl)-5,7-dimethoxypyrido[2,3-d]pyrimidin-4(3H)-one;
5,7-dimethoxy-2-(4-methoxy-3-(morpholinomethyl)phenyl)quinazolin-4(3H)-one;
2-(3,5-dimethyl-4-(2-(pyrrolidin-1-yl)ethoxy)phenyl)-5,7-dimethoxyquinazolin-4(3H)-one;
2-(4-hydroxy-3-(2-hydroxyethyl)phenyl)-5,7-dimethoxyquinazolin-4(3H)-one;
2-(4-(bis(2-hydroxyethyl)amino)phenyl)-5,7-dimethoxy-pyrido[2,3-d]pyrimidin-4(3H)-one;
5,7-dimethoxy-2-(4-(2-methoxyethoxy)-3,5-dimethylphenyl)quinazolin-4(3H)-one;
2-(4-(2-hydroxyethoxy)-3,5-dimethylphenyl)-5-methoxyquinazolin-4(3H)-one;
(E)-N′-4-(5,7-dimethoxy-4-oxo-3,4-dihydroquinazolin-2-yl)-2,6-dimethylphenyl)-N,N-dimethylformimidamide;
2-(4-(benzyloxy)-3,5-dimethylphenyl)-5,7-dimethoxyquinazolin-4(3H)-one;
2-(4-(2-aminoethoxy)-3,5-dimethylphenyl)-5,7-dimethoxyquinazolin-4(3H)-one;
2-(4-(5,7-dimethoxy-4-oxo-3,4-dihydroquinazolin-2-yl)-2-methoxyphenoxy)acetic acid;
2-(4-(5,7-dimethoxy-4-oxo-3,4-dihydroquinazolin-2-yl)-2,6-dimethyl-phenoxy)ethyl propylcarbamate;
2-(4-(5,7-dimethoxy-4-oxo-3,4-dihydroquinazolin-2-yl)-2,6-dimethyl-phenoxy)ethyl methylcarbamate;
N-(2-(4-(5,7-dimethoxy-4-oxo-3,4-dihydroquinazolin-2-yl)-2,6-dimethylphenoxy)ethyl)-4-methylbenzamide;
N-(2-(4-(5,7-dimethoxy-4-oxo-3,4-dihydroquinazolin-2-yl)-2,6-dimethylphenoxy)ethyl)benzenesulfonamide;
N-(2-(4-(5,7-dimethoxy-4-oxo-3,4-dihydroquinazolin-2-yl)-2,6-dimethylphenoxy)ethyl)-4-methylbenzenesulfonamide;
N-(2-(4-(5,7-dimethoxy-4-oxo-3,4-dihydroquinazolin-2-yl)-2,6-dimethylphenoxy)ethyl)-4-methoxybenzamide;
N-(2-(4-(5,7-dimethoxy-4-oxo-3,4-dihydroquinazolin-2-yl)-2,6-dimethylphenoxy)ethyl)benzamide;
1-(2-(4-(5,7-dimethoxy-4-oxo-3,4-dihydroquinazolin-2-yl)-2,6-dimethylphenoxy)ethyl)-3-methylurea;
1-(2-(4-(5,7-dimethoxy-4-oxo-3,4-dihydroquinazolin-2-yl)-2,6-dimethylphenoxy)ethyl)-3-(4-methoxyphenyl)urea;
2-(3,5-dimethyl-4-(2-morpholinoethoxy)phenyl)quinazolin-4(3H)-one;
2-(4-(2-hydroxyethoxy)-3,5-dimethylphenyl)quinazolin-4(3H)-one;
2-(2,3-dihydrobenzo[b][(1,4]dioxin-6-yl)quinazolin-4(3H)-one;
2-(4-(4-oxo-3,4-dihydroquinazolin-2-yl)phenoxy)acetic acid;
2-(4-(dimethylamino)naphthalen-1-yl)quinazolin-4(3H)-one;
2-(4-(4-oxo-3,4-dihydroquinazolin-2-yl)phenoxy)acetamide;
2-(4-(bis(2-hydroxyethyl)amino)phenyl)quinazolin-4(3H)-one;
2-(4-(5,7-dimethoxyquinazolin-2-yl)-2,6-dimethylphenoxy)ethanol;
2-(4-(2-hydroxyethoxy)-3,5-dimethylphenyl)-5,7-dimethylquinazolin-4(3H)-one;
5,7-dichloro-2-(4-(2-hydroxyethoxy)-3,5-dimethylphenyl)quinazolin-4(3H)-one;
6-bromo-2-(4-hydroxy-3,5-dimethylphenyl)quinazolin-4(3H)-one;
6-bromo-2-(4-(2-hydroxyethoxy)-3,5-dimethylphenyl)quinazolin-4(3H)-one;
6-bromo-2-(4-(2-(tert-butyldimethylsilyloxy)ethoxy)-3,5-dimethylphenyl)quinazolin-4(3H)-one;
5,7-dimethoxy-2-(pyridin-4-yl)quinazolin-4(3H)-one;
2-(4-(dimethylamino)naphthalen-1-yl)-5,7-dimethoxyquinazolin-4(3H)-one;
2-(4-(dimethylamino)pyridin-1-yl)-6,7-dimethoxyquinazolin-4(3H)-one;
2-(4-hydroxy-3,5-dimethylphenyl)-5,7-dimethoxy-1-methylquinazolin-4(3H)-one;
2-(4-hydroxy-3,5-dimethylphenyl)-5,7-dimethoxy-6-(morphoinomethyl)quinazolin-4(3H)-one;
2-(4-(2-hydroxyethoxy)-3,5-dimethylphenyl)-6-methoxyquinazolin-4(3H)-one;
5-hydroxy-2-(4-hydroxy-3,5-dimethylphenyl)-7-methoxyquinazolin-4(3H)-one;
3-(4-hydroxy-3,5-dimethylphenyl)-6,8-dimethoxyisoquinolin-1(2H)-one;
3-(4-(2-hydroxyethoxy)-3,5-dimethylphenyl)-6,8-dimethoxyisoquinolin-1(2H)-one;
2-(4-hydroxy-3,5-dimethylphenyl)-5,7-dimethoxyquinazolin-4(3H)-one;
2-(4-(2-hydroxyethoxy)-3,5-dimethylphenyl)-5,7-dimethoxyquinazolin-4(3H)-one);
2-(4-hydroxy-3-methoxyphenyl)-5,7-dimethoxyquinazolin-4(3H)-one;
2-(2,3-dihydrobenzo[b][1,4]dioxin-6-yl)-5,7-dimethoxyquinazolin-4(3H)-one;
2-(4-(bis(2-hydroxyethyl)amino)phenyl)-5,7-dimethoxyquinazolin-4(3H)-one;
2-(4-(bis(2-hydroxyethyl)amino)phenyl)-6,7-dimethoxyquinazolin-4(3H)-one;
2-(2,3-dihydrobenzo[b][1,4]dioxin-6-yl)-6,7-dimethoxyquinazolin-4(3H)-one;
2-(4-((4-ethylpiperazin-1-yl)methyl)phenyl)-5,7-dimethoxyquinazolin-4(3H)-one;
2-(4-(2-hydroxyethoxy)-3,5-dimethylphenyl)-5,7-dimethoxypyrido[2,3-d]pyrimidin-4(3H)-one;
2-(2-chloro-6-methylpyridin-4-yl)-5,7-dimethoxyquinazolin-4(3H)-one;
5,7-dimethoxy-2-(4-methoxy-3,5-dimethylphenyl)quinazolin-4(3H)-one;
2-(4-amino-3, 5-dimethylphenyl)-5,7-dimethoxyquinazolin-4(3H)-one;
N1-(2-(4-(5,7-dimethoxy-4-oxo-3,4-dihydroquinazolin-2-yl)-2,6-dimethylphenoxy)ethyl)-N2-methylphthalamide;
4-chloro-N-(2-(4-(5,7-dimethoxy-4-oxo-3,4-dihydroquinazolin-2-yl)-2,6-dimethylphenoxy)ethyl)benzenesulfonamide;
3-(4-(5,7-dimethoxy-4-oxo-3,4-dihydroquinazolin-2-yl)phenyl)propanoic acid;
5,7-dimethoxy-2-(4-((4-methylpiperazin-1-yl)methyl)phenyl)quinazolin-4(3H)-one;
5,7-dimethoxy-2-(4-(morpholinomethyl)phenyl)quinazolin-4(3H)-one;
5,7-dimethoxy-2-(4-morpholinophenyl)quinazolin-4(3H)-one;
2-(4-(5,7-dimethoxy-4-oxo-3,4-dihydroquinazolin-2-yl)-2,6-dimethylphenoxy)ethyl cyclohexylcarbamate;
N-(2-(4-(5,7-dimethoxy-4-oxo-3,4-dihydroquinazolin-2-yl)-2,6-dimethylphenoxy)ethyl)acetamide;
N-(2-(4-(5,7-dimethoxy-4-oxo-3,4-dihydroquinazolin-2-yl)-2,6-dimethylphenoxy)ethyl)isobutyramide;
1-(2-(4-(5,7-dimethoxy-4-oxo-3,4-dihydroquinazolin-2-yl)-2,6-dimethylphenoxy)ethyl)-3-phenylurea; and
3-(2-(4-(5,7-dimethoxy-4-oxo-3,4-dihydroquinazolin-2-yl)-2,6-dimethylphenoxy)ethyl-1,1-dimethylurea.
